(19) United States
(12) Patent Application Publication
(10) Pub. No.: US 2003/0167049 A1
(43) Pub. Date: Sep. 4, 2003

(54) Disposable absorbent garment with adjustable side panels

(76) Inventor: Bernadette M. Gibbs, Statham, GA (US)

Correspondence Address: Hunton & Williams, 1900 K Street, N.W., Washington, DC, 20006-1109, US

(21) Appl. No.: 10/086,541

(22) Filed: Mar. 4, 2002

(51) Int. Cl. A61F 13/15

(52) U.S. Cl. 604/386; 604/391

(57) ABSTRACT

An absorbent article is provided that comprises a main body having a first main body waist portion, a second main body waist portion and a crotch region disposed between the first and second main body waist portions. The main body has orthogonal longitudinal and lateral axes. The absorbent article further comprises a pair of side panels, each side panel connecting the first main body waist portion to the second main body waist portion. The first main body waist portion, the second main body waist portion and the side panels collectively define a waist opening edge and two leg opening edges. Each side panel is formed from a first side portion extending laterally outward from the first main body waist portion and a second side portion extending laterally outward from the second main body waist portion. The first side portion is attached to the second side portion by a side seam intersecting the waist opening edge at a first seam angle and intersecting one of the leg openings at a second seam angle when the first and second side portions are in a fully stretched condition. The absorbent article also comprises an adjustment arrangement including at least one adjustment tab associated with one of the side panels. Each of the at least one adjustment tab includes the side seam of the associated side panel and extends outward therefrom. Each of the at least one adjustment tab has a lateral tab edge. The adjustment arrangement also includes at least one adjustment tab fastener adapted for removably attaching the at least one adjustment tab to a receiving portion of the main body.

### BACKGROUND OF THE INVENTION

1. Field of the Invention

The invention relates generally to the field of absorbent garments, and more particularly to a disposable absorbent garment that is adjustable for a comfortable, secure fit.

2. Description of Related Art

Disposable absorbent garments such as infant diapers or training pants, adult incontinence products, and other such products are well-known in the art. Typically, these garments comprise a main body formed from a liquid-permeable body-contacting liner sheet (or “topsheet”), a liquid-impermeable backing sheet (or “backsheet”) (collectively the “sheets”), and a moisture-absorbent core fiber (or “absorbent core”) that usually is made of a mat of randomly arrayed cellulose fiber and is generally disposed between the topsheet and the backsheet.

One type of disposable absorbent garment is the infant diaper, which is typically a flat, open-sided garment that is fitted about a child with the child laying down on top of the garment. The back portion of the garment is typically releasably connected to the front portion of the garment by a closure mechanism or fastener. Diapers are thus typically meant for use by children dependent upon a parent for assistance in putting on the absorbent garment.

Disposable diapers of this type are often replaced with pants-type diapers, or training pants, for older children who are able to partially dress themselves but still require absorbent undergarments. These pants-type diapers have closed sides and do not require a closure mechanism at the front or back of the garment. Because they are similar to conventional underwear, they can be put on and taken off with the child in a standing position. The construction of such garments is such that the garments can be easily donned by the child without assistance. This helps the child to make the transition between diapers and conventional underpants. Moreover, training pants are generally designed so that security against leakage can be maintained without parental adjustment.

Pant-like disposable absorbent garments are also available for adults. While the size and relative proportions of the components of these garments are different from the training pants garments, the overall construction is generally similar. In both types, the main body is doubled to provide front and back panels. The upper portions of the front and back panels are connected by stretchable side panels attached to the lateral edges of the main body. Leg holes are formed by the lower portion of the doubled body and the stretchable side panels. Typically, elastic leg gathers are provided to cause the garment to conform to the legs of the wearer thereby preventing leakage around the leg holes.

The configuration of the components of disposable absorbent garments are often driven in part by manufacturing methods wherein the garments are assembled in a continuously moving assembly line. The efficiency of these methods is dependent on the ability to continuously provide, place and attach the various components. This often requires that the designer of the garment consider such parameters as the speed and direction of motion of the garment during assembly and minimize the number of intermediate manufacturing steps that must be performed on the various components. These considerations have, at times, caused the sacrifice of features that could enhance the fit and comfort of the garments.

### SUMMARY OF THE INVENTION

The present invention provides an absorbent article comprising a main body having a first main body waist portion, a second main body waist portion and a crotch region disposed between the first and second main body waist portions. The main body has orthogonal longitudinal and lateral axes. The absorbent article further comprises a pair of side panels, each side panel connecting the first main body waist portion to the second main body waist portion. The first main body waist portion, the second main body waist portion and the side panels collectively define a waist opening edge and two leg opening edges. Each side panel is formed from a first side portion extending laterally outward from the first main body waist portion and a second side portion extending laterally outward from the second main body waist portion. The first side portion is attached to the second side portion by a side seam intersecting the waist opening edge at a first seam angle and intersecting one of the leg openings at a second seam angle when the first and second side portions are in a fully stretched condition. The absorbent article also comprises an adjustment arrangement including at least one adjustment tab associated with one of the side panels. Each of the at least one adjustment tab includes the side seam of the associated side panel and extends outward therefrom. Each of the at least one adjustment tab has a lateral tab edge. The adjustment arrangement also includes at least one adjustment tab fastener adapted for removably attaching the at least one adjustment tab to a receiving portion of the main body.

These and other objects, features and advantages of the invention will be apparent through the detailed description of the preferred embodiments and the drawings attached hereto. It also is to be understood that both the foregoing general description and the following detailed description are exemplary and explanatory and are not restrictive of the scope of the invention.

### BRIEF DESCRIPTION OF THE DRAWINGS

The invention may be understood more readily by viewing the drawings, in which:

FIG. 1 is a perspective view of an absorbent garment according to an embodiment of the invention as it would appear when worn by a user in an unadjusted configuration;

FIG. 2 is a perspective view of the absorbent garment of FIG. 1 as it would appear when worn by a user in an adjusted configuration;

FIG. 3 is a front view of an absorbent garment according to an embodiment of the invention;

FIG. 4 is a partial cutaway view of an unfinished absorbent garment according to an embodiment of the invention, the garment being laid flat as it would appear during an intermediate stage of manufacture;

FIG. 5 is a perspective view of an absorbent garment according to an embodiment of the invention as it would appear when worn by a user in an unadjusted configuration;

FIG. 6 is a perspective view of the absorbent garment of FIG. 6 as it would appear when worn by a user in an adjusted configuration;

FIG. 7 is a front view of an absorbent garment according to an embodiment of the invention;

FIG. 8 is a partial cutaway view of an unfinished absorbent garment according to an embodiment of the invention, the garment being laid flat as it would appear during an intermediate stage of manufacture;

FIG. 9 is a front view of an absorbent garment according to an embodiment of the invention;

FIG. 10 is a perspective view of an absorbent garment according to an embodiment of the invention as it would appear when worn by a user in an adjusted configuration;

FIG. 11 is a front view of an absorbent garment according to an embodiment of the invention;

FIG. 12 is a perspective view of an absorbent garment according to an embodiment of the invention as it would appear when worn by a user in an adjusted configuration;

FIG. 13 is a front view of an absorbent garment according to an embodiment of the invention;

FIG. 14 is a perspective view of an absorbent garment according to an embodiment of the invention as it would appear when worn by a user in an adjusted configuration;

FIG. 15 is a front view of an absorbent garment according to an embodiment of the invention;

FIG. 16 is a perspective view of an absorbent garment according to an embodiment of the invention as it would appear when worn by a user in an adjusted configuration;

FIG. 17 is a front view of an absorbent garment according to an embodiment of the invention;

FIG. 18 is a perspective view of an absorbent garment according to an embodiment of the invention as it would appear when worn by a user in an adjusted configuration;

FIG. 19 is a front view of an absorbent garment according to an embodiment of the invention;

FIG. 20 is a perspective view of an absorbent garment according to an embodiment of the invention as it would appear when worn by a user in an adjusted configuration;

FIG. 21 is a front view of an absorbent garment according to an embodiment of the invention;

FIG. 22 is a perspective view of an absorbent garment according to an embodiment of the invention as it would appear when worn by a user in an adjusted configuration;

FIG. 23 is a perspective view of an absorbent garment according to an embodiment of the invention as it would appear when worn by a user in an unadjusted configuration;

FIG. 24 is a perspective view of the absorbent garment of FIG. 23 as it would appear when worn by a user in an adjusted configuration; and

FIG. 25 is a front view of an absorbent garment according to an embodiment of the invention.

### DETAILED DESCRIPTION OF THE INVENTION

The present invention provides a pant-like absorbent garment with stretchable side panels that are adjustable so as to better conform to the body of the wearer, thereby providing a more comfortable fit. As used herein, the terms “absorbent garment” and “absorbent garment” refer to garments that absorb and contain exudates, and more specifically, refer to garments that are placed against or in proximity to the body of the wearer to absorb and contain the various exudates discharged from the body. A non-exhaustive list of examples of absorbent garments includes diapers, diaper covers, disposable diapers, training pants, feminine hygiene products, and adult incontinence products. The term “disposable absorbent garment” refers to absorbent garments that are intended to be discarded or partially discarded after a single use (i.e., they are not intended to be laundered or otherwise restored or reused).

The present invention can be used with all of the foregoing classes of absorbent garments, without limitation, whether disposable or otherwise. These classifications are used interchangeably throughout the specification, but are not intended to limit the claimed invention. The invention will be understood to encompass, without limitation, all classes and types of absorbent garments, including those described above.

The term “component,” can refer, but is not limited, to designated selected regions, such as edges, corners, sides or the like; structural members, such as elastic strips, absorbent pads, stretchable layers or panels, layers of material, or the like; or a graphic.

Absorbent garments and diapers may have a number of different constructions. In each of these constructions it is generally the case that an absorbent core is disposed between a liquid pervious, body-facing topsheet, and a liquid impervious, exterior backsheet. In some cases, one or both of the topsheet and backsheet may be shaped to form a pant-like garment. In other cases, the topsheet, backsheet and absorbent core may be formed as a discrete assembly that is placed on a main chassis layer and the chassis layer is shaped to form a pant-like garment. The garment may be provided to the consumer in the fully assembled pant-like shape, or may be partially pant-like and require the consumer to take the final steps necessary to form the final pant-like shape. In the case of training pant-type garments and most adult incontinent products, the garment is provided fully formed with factory-made side seams and the garment is donned by pulling it up the wearer's legs. In the case of diapers, a caregiver usually wraps the diaper around the wearer's waist and joins the side seams manually by attaching one or more adhesive or mechanical tabs, thereby forming a pant-like structure. For clarity, the present invention is described herein only with reference to a training pant-type garment in which the topsheet, backsheet and absorbent core are assembled into a main body that, when combined with side panels forms a pant-like garment, although the invention may be used with other constructions.

Throughout this description, the term “disposed” and the expressions “disposed on,” “disposing on,” “disposed in,” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “topsheet” and “backsheet” denote the relationship of these materials or layers with respect to an absorbent core. It is understood that additional layers may be present between the absorbent core and the topsheet and backsheet, and that additional layers and other materials may be present on the side opposite the absorbent core from either the topsheet or the backsheet.

The present invention provides an absorbent garment having a main body formed with a topsheet and a backsheet having an absorbent core disposed therebetween. The main body is a generally planar structure that is doubled to form a front main body waist portion and a rear main body waist portion connected by a crotch region. The front main body waist portion is connected to the rear main body waist portion by a pair of side panels. The front main body waist portion, the rear main body waist portion and the side panels collectively define a waist opening surrounded by a waist opening edge and two leg openings each surrounded by a leg opening edge.

Each side panel of the absorbent garment may be formed from a generally planar side panel member that is attached to the main body along a lateral edge of the main body and is doubled along with the main body. Alternatively, the side panel members may be integrally formed with the backsheet or other component of the main body. The side panel members may each have a relatively narrow central portion adjacent the crotch region of the main body and wider front and rear portions adjacent the front and rear main body waist portions, respectively. The front portion of each side panel member is joined to the rear portion of the side panel by forming a side panel seam that runs from the waist opening edge to one of the leg opening edges. The side panel seam may be angled inward adjacent the intersection of the seam with the waist opening edge, adjacent the intersection of the seam with the leg opening edge or adjacent both intersections. At least a portion of the side seam may be curved to enhance the comfort and fit of the disposable garment as is described in co-pending U.S. patent application Ser. No. 10/074,028, filed Feb. 14, 2002 (attorney docket no. 53394.000566), which is incorporated herein by reference in its entirety.

Absorbent garments according to embodiments of the present invention may include an adjustment arrangement operatively associated with the side panels of the garment. The adjustment arrangement is configured to allow the adjustment of the side panels for better conformance to the body contour of the wearer, thereby providing enhanced comfort and leakage security. The adjustment arrangement may include one or more adjustment tabs extending laterally outward from each side seam of the garment. The adjustment tabs are arranged so that when the lateral ends of the tabs are placed in tension and drawn toward the front (or rear) main body waist portion, the side panel, along with the waist opening, the leg opening on the side of the adjustment tab or both the waist opening and the leg opening, is tightened to more closely conform to the body contour of the wearer. One or more adjustment tab fasteners are provided to allow the adjustment tab to be removably attached to the main body.

Those skilled in the art will recognize that “front” and “rear” in the context of the invention denote for clarity purposes only the front and rear of a user, and that the absorbent garment could be reversed whereby the previously described “front” portion becomes the rear portion, and vice versa.

The invention will now be described with reference to the attached drawings, which illustrate various embodiments of the invention. For clarity, features that appear in more than one figure have the same reference number in each figure.

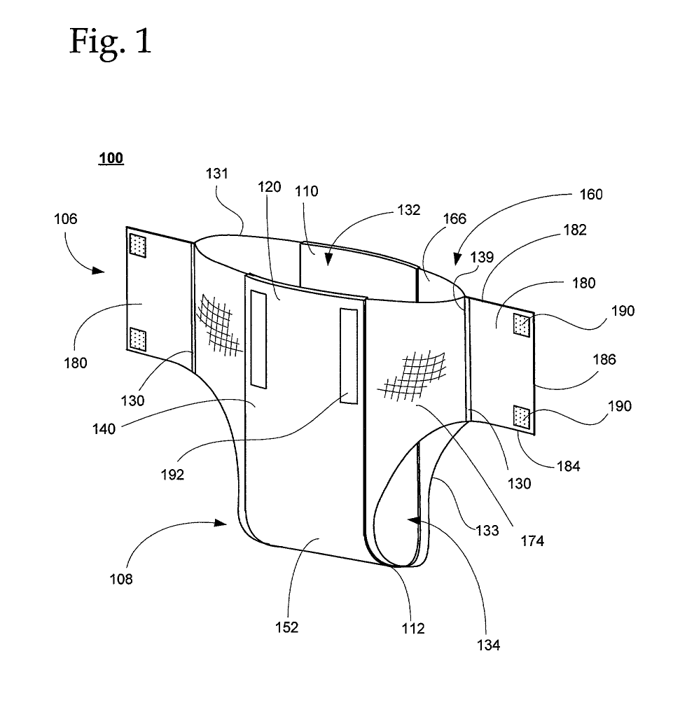

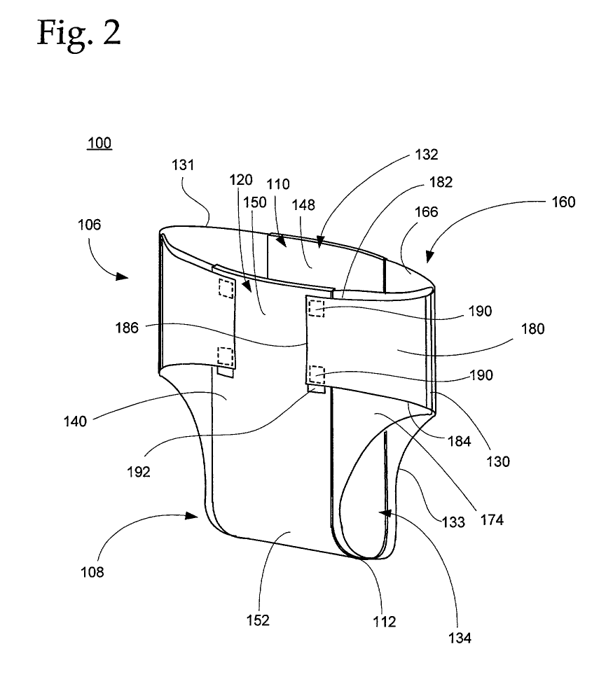

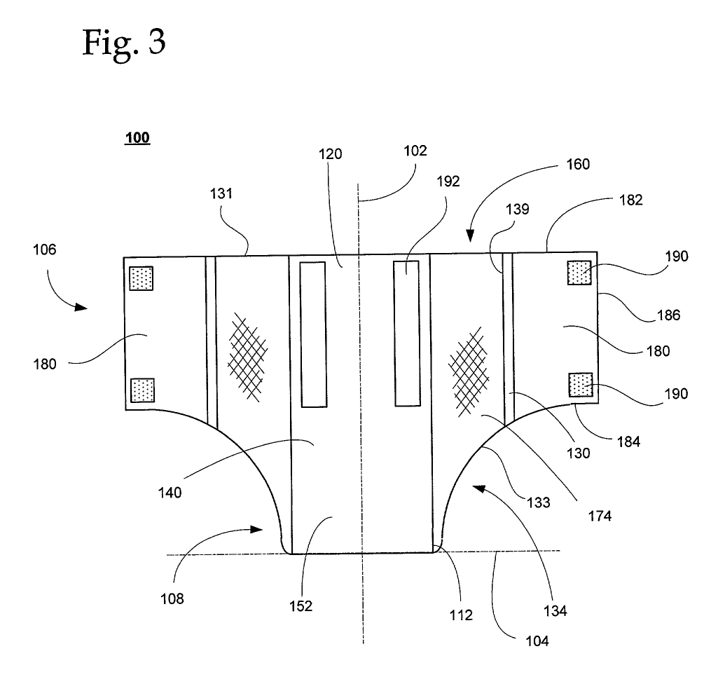

FIGS. 1-3 depict a preferred embodiment of an absorbent garment (preferably a disposable absorbent garment) 100 of the present invention. The absorbent garment 100 is depicted as and will be discussed as a child's training pant; however, this depiction is not intended to limit the invention, and those skilled in the art will appreciate that the invention covers other types of absorbent garments including incontinent pants for adults. The absorbent garment 100 is depicted in FIGS. 1-3 in its fully assembled form as it would appear when worn, with FIGS. 1 and 2 being perspective views and FIG. 3 a front view.

Unless otherwise noted, in all drawings included herein, the elastic components of garments of the various embodiments of the invention are depicted in their relaxed condition with the effects of the elastics removed for clarity (when relaxed, the elastics typically cause the surrounding material to gather or “shirr”).

In use, the garment 100 is a pant-like structure having a waist-encircling region 106 and a crotch region 108. The waist-encircling region 106 may include a first waist portion 110, disposable adjacent to, for example, the back waist area of a wearer's body, and a second waist portion 120, disposable adjacent to, for example, the front waist area of a wearer's body. The first and second waist portions 110, 120 may therefore correspond to the back and front of the wearer's body, respectively. The first and second waist portions 110, 120 are joined to one another at side seams 130 to form the waist encircling portion 106 and to define a waist opening edge 131 surrounding a waist opening 132 and leg opening edges 133 surrounding two leg openings 134.

FIGS. 1 and 3 illustrate the garment 100 as it would appear when initially donned by the wearer. The garment 100 is shown with and will be described relative to first and second orthogonal axes 102, 104. The first axis 102 may be referred to as the vertical axis 102 and the second axis 104 may be referred to as the lateral axis 104. It will be understood by those having ordinary skill in the art that the vertical axis 102 corresponds generally to a head-to-toe axis of a standing wearer and that the lateral axis 104 corresponds generally to a side-to-side axis of a standing wearer.

The garment 100 includes a generally planar main body 140 doubled to form a generally U-shaped structure. The main body 140 has a first main body waist portion 148 configured to be disposable adjacent the back of the wearer's waist area and a second main body waist portion 150 configured to be disposable adjacent the front of the wearer's waist area. The main body 140 also has a central main body portion 152 that connects the first and second main body waist portions 148, 150 and forms at least a portion of the crotch region 108. The first and second main body waist portions 148, 150 are connected on each lateral side of the garment by a side panel 160. Each side panel 160 preferably has a first side portion 166 attached to a lateral edge 168 of the main body 140 adjacent the first main body waist portion 148 and a portion of the central main body portion 152. Each side panel 160 also preferably has a second side portion 174 attached to the main body lateral edge 168 adjacent the second main body waist portion 150 and a portion of the central main body portion 152. The first and second side portions 166, 174 are joined at a side seam 130. The first and second side seam portions 166, 174 are joined and the side seam 130 is formed with the first and second side seam portions 166, 174 in a fully stretched condition.

The side seams 130 of the garment 100 are formed in a generally straight line that is preferably substantially vertical (i.e., is within about 5 degrees of being parallel to the vertical axis 102). Although it is preferable in this embodiment that the seam 130 be substantially parallel to the vertical axis, it will be understood that other orientations may be used without departing from the scope of the invention. As will be discussed, other side seam geometries may also be used.

As used herein the term “seam” indicates an area of contact between two surfaces wherein the contact is maintained by a mechanical, chemical, adhesive or other type of bond formed along a substantially continuous path. The side seam 130 may be formed using any method known in the art or a combination of such methods. Examples of such methods include: adhesive bonding using hot melt adhesives, construction adhesives or both, chemical or solvent bonding, stitching, heat bonding, autogenous bonding, and ultrasonic welding. Each of these methods may be used to form a seam having a predetermined geometric or free-form path.

The first and second main body waist portions 148, 150 combine with the side panels 160 to form the waist opening edge 131 and the waist opening 132. The central main body portion 152 combines with the side panels 160 to form the leg opening edges 133 and the leg openings 134. The side panels 160 are preferably formed from an elastic or elasticized material to allow the waist opening 132 and the leg openings 134 to expand and contract to provide a secure, leak-free fit.

The garment 100 includes an adjustment arrangement configured to allow the adjustment of the side panels 160 to better conform to the body shape of the wearer. The adjustment arrangement preferably includes a pair of adjustment tabs 180 that include the respective side seams on either side of the garment and that extend laterally outward from the inner edges 139 of the side seams 130. The adjustment tabs 180 are configured so that they can be grasped and pulled outward and then toward the first main body waist portion 148 (or the second main body waist portion 150) where they may be removably connected to the main body 140 using a fastening arrangement.

The fastening arrangement may include one or more gripping members 190 attached directly to the adjustment tab 180 as shown in FIGS. 1-3. The gripping members 190 are configured to releasably attach the adjustment tabs 180 to a receiving portion 192 on the main body 140. The gripping members 190 may each comprise a hook surface such as the hook portion of a hook-and-loop fastener, the hook surface being adapted for reversible mating engagement with the receiving portion 192. The receiving portion 192 may include a corresponding loop surface such as the loop portion of a hook-and-loop portion. Alternatively, some or all of the backsheet used to form the main body 140 may be formed using a material that is grippable by the hook surface. In another embodiment, the gripping member 190 may comprise a releasable pressure sensitive adhesive for releasably adhering the adjustment tab 180 to the receiving portion 192 of the main body 140. A protective release cover may be applied to the adhesive surface to protect the adhesive surface from contamination prior to use. The release cover may be removed just prior to use of the adjustment tabs 180 to adjust the fit of the garment 100.

One or more gripping members 190 are preferably positioned adjacent the lateral tab edge 186 of the adjustment tab 180. A gripping member 190 may be positioned adjacent an upper tab edge 182 for securing an adjustment of the waist opening 132. Alternatively or in addition, a gripping member 190 may be positioned adjacent a lower tab edge 184 for securing an adjustment of one of the leg openings 134. In an alternative embodiment, a single elongated gripping member may be oriented along the lateral tab edge 186 that extends from the area adjacent the upper tab edge 182 to the area adjacent the lower tab edge 184.

The fastening arrangement serves to secure the adjustment tabs 180 in place while maintaining tension on the side seams 130. In an embodiment having two gripping members 190 at the corners of the adjustment tab 180, this may result in a fastened configuration such as that shown in FIG. 2. In the fastened configuration of FIG. 2, the first side portion 166 is placed in tension while the second side portion is folded back along the side seam 130. This has the effect of snugging the garment in place against the body of the wearer. It also has the effect of reducing the circumference of the waist opening 132 and associated leg opening 134.

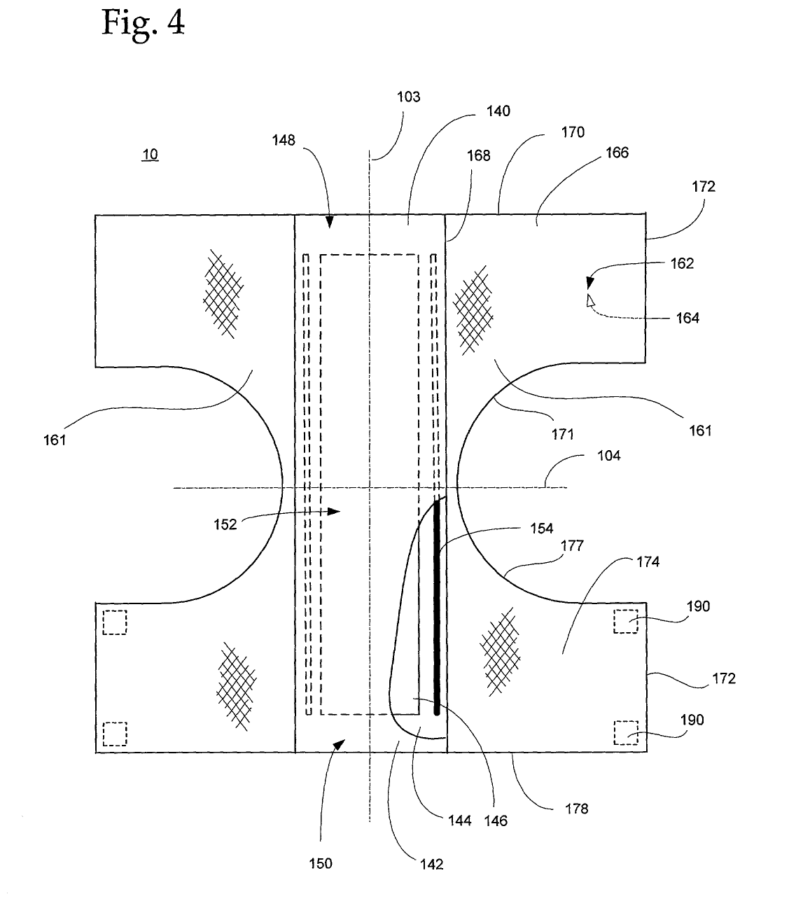

The components of the absorbent garment 100 are preferably assembled into a generally planar structure that can be doubled to form the U-shaped structure illustrated in FIGS. 1-3. FIG. 4 illustrates an unfinished garment 10 that can be used as a precursor to the absorbent final garment 100. At this intermediate stage of construction, the unfinished garment 10 is fully assembled except for the formation of the side seams. Several different final embodiments may be constructed from the unfinished garment 10, including the absorbent garment 100 of FIGS. 1-3. FIG. 4 is a plan view of the body-facing side of the unfinished garment 10 in a generally flattened condition. The unfinished garment 10 may have a generally hourglass shaped structure, but it may also have any other shape suitable for the given application.

The flattened unfinished garment 10 has a longitudinal axis 103 extending from the portion of the garment 10 that will conform to the front of the user to the portion of the garment 10 that will conform to the rear of the user. The longitudinal axis of the flattened garment 10 is orthogonal to the lateral axis 104 that extends from side to side relative to a wearer of the garment 100.

The unfinished garment 10 has a main body 140 that preferably comprises a topsheet 142 and a backsheet 144 that may be substantially coterminous with the topsheet 142. Alternatively, the backsheet may be used as a base sheet upon which the other main body components are built. In such embodiments, the backsheet may extend laterally outward beyond the lateral edges 168 of the main body 140. When the completed garment is worn, the topsheet 142 faces the wearer's body, and the backsheet 144 faces away from the wearer's body. An absorbent core 146 preferably is disposed between at least a portion of the topsheet 142 and the backsheet 144. As discussed above with respect to the completed garment 100, the main body 140 has first and second main body waist portions 148, 150 connected by a central main body portion 152.

The topsheet 142 and backsheet 144 may be constructed from a wide variety of materials known in the art. The invention is not intended to be limited to any specific materials for these components. The topsheet 142 and backsheet can be shaped and sized according to the requirements of various types of absorbent garments, or to accommodate various user sizes. The topsheet 142 and backsheet 144 may, for example be configured to provide a main body 140 that is hourglass shaped, rectangular, trapezoidal, “T” shaped, or other geometry.

Embodiments of the present invention may further comprise various additional features. For example, one or more pairs of elastic gathers 154 may extend adjacent the lateral edges 168 of the main body 140. The main body 140 may also comprise one or more waste containment systems, such as inboard standing leg gathers (not shown), which preferably extend from the second main body waist portion 150 to the first main body waist portion 148 along opposite sides of longitudinal center line 103. One or both of the first and second main body waist portions 148,150 may also be equipped with strips of elastic waist foam or other elastically extensible material (not shown), which help contract the garment around the wearer's waist, providing improved fit and leakage prevention.

The unfinished garment 10 is provided with side panels 160 extending laterally outward from the lateral edges 168 of the main body 140. Each side panel 160 may be formed using a single continuous piece of elastic material having a body facing surface 162 and an opposing outward facing surface 164 as shown in FIG. 4. Alternatively, the side panel 160 may be formed from multiple portions of elastic material. For example, one portion of material could be used in the area adjacent the first main body waist portion 148 and a second portion of material could be used in the area adjacent the second main body waist portion 150. It will be understood that if multiple portions of elastic material are used to form the side panel 160, the side panel 160 could be formed without elastic material adjacent the central main body portion 152.

Each side panel 160 preferably has a first side portion 166 attached to a lateral edge 168 of the main body 140 adjacent the first main body waist portion 148 and a portion of the central main body portion 152. The first side portion 166 has a waist opening edge 170 and a lateral side panel edge 172. Each side panel 160 also has a second side portion 174 attached to the main body lateral edge 168 adjacent the second main body waist portion 150 and a portion of the central main body portion 152. The second side portion 174 has a waist opening edge 178 and a lateral side panel edge 179.

In some embodiments of the invention, the first and second side portions 166, 174 may be integrally formed with one or more components of the main body 140. In an exemplary preferred embodiment, the first and second side portions 166, 174 are formed by portions of the backsheet 144 that extend laterally outward beyond the lateral edges 168 of the main body 140.

The first and second side portions 166, 174 are configured so that the body facing surface 162 in the area adjacent the lateral side panel edge 172 of the first side portion 166 may be made to engage the body facing surface 162 in the area adjacent the lateral side panel edge 179 of the second side portion 174 when the garment 10 is folded to convert it from the configuration of FIG. 4 to a final assembled configuration. With the body facing surface 162 of the first and second side portions 166, 174 so-engaged and in a fully stretched condition, the first and second side portions 166, 174 are joined by the formation of side seam 130 thereon.

The formation of the side seam 130 results in the formation of a side panel from the first and second side portions 166, 174. By forming a straight, substantially vertical side seam 130 on each side panel 160 of the unfinished garment 10, a completed absorbent garment 100 may be formed. As will be discussed, other garment embodiments may be formed from the unfinished garment 10 by forming side seams having a different geometry. The areas of the first and second side portions 166, 174 that are lateral to the side seam 130 may be joined in any suitable fashion to form the adjustment tabs 180. Additional materials may be added to these areas to enhance the strength of the adjustment tab 180. In an alternative embodiment, one of the side portions may be cut adjacent the side seam 130, leaving the other side portion for use in forming the adjustment tab 180. In still another embodiment, the adjustment tab 180 may be formed by attaching a separate sheet or composite material to the side portions 166, 174 at or adjacent the side seam 130.

In embodiments where the second side portion 174 is used to form the adjustment tab 180, gripping members 190 may be attached to the outward facing surface 164 of the second side portion 174. This may be accomplished either before or after the side seam 130 is formed. It will be understood that if the first side portion 166 is used to form the adjustment tab 180 but the second side portion 174 is not, the gripping members 190 may be attached to the body facing surface 162 of the first side portion.

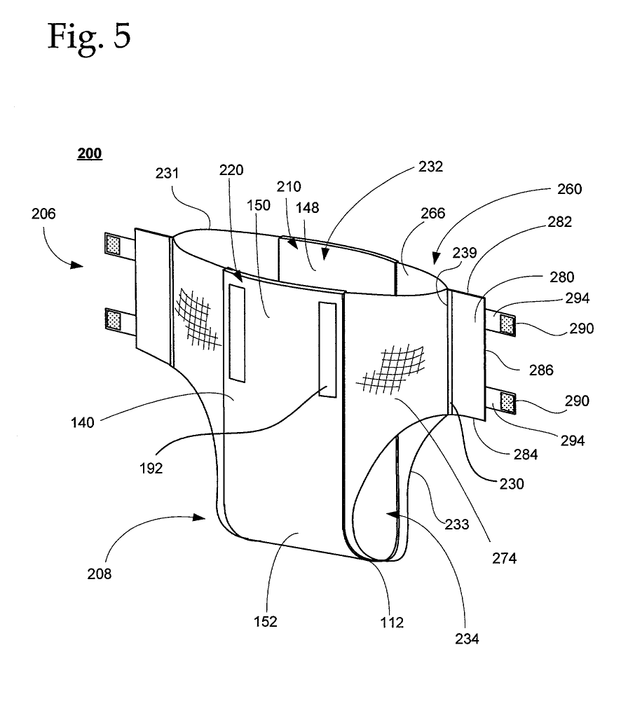

FIGS. 5-7 illustrate an absorbent garment 200 that is similar to the absorbent garment 100 except that the adjustment tabs each use an attachment arrangement with a pair of attachment strips that extend laterally outward from the lateral edge of the adjustment tab. This allows the use of smaller adjustment tabs and, accordingly, less elastic material in the side portions used to form the side panels and the adjustment tabs. The absorbent garment 200 has a waist-encircling region 206 and a crotch region 208. The waist-encircling region may include a first waist portion 210, disposable adjacent to the back waist area of a wearer's body, and a second waist portion 220, disposable adjacent to the front waist area of a wearer's body. The first and second waist portions 210, 220 may therefore correspond to the back and front of the wearer's body, respectively. The first and second waist portions 210, 220 are joined to one another at side seams 230 to form the waist encircling portion 206 and to define a waist opening edge 231 surrounding a waist opening 232 and leg opening edges 233 surrounding two leg openings 234.

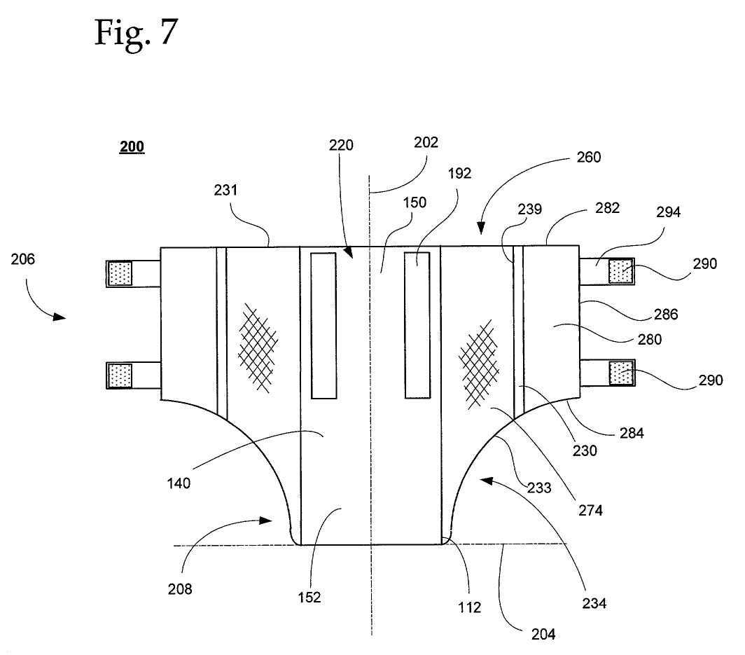

FIGS. 5 and 7 illustrate the garment 200 as it would appear when initially donned by the wearer. The garment 200 is shown with and will be described relative to first and second orthogonal axes 202, 204. The first axis 202 may be referred to as the vertical axis 202 and the second axis 204 may be referred to as the lateral axis 204. It will be understood by those having ordinary skill in the art that the vertical axis 202 corresponds generally to a head-to-toe axis of a standing wearer and that the lateral axis 204 corresponds generally to a side-to-side axis of a standing wearer.

As shown in FIGS. 5-7, the absorbent garment 200 includes a main body 140 that is substantially similar to that of the absorbent garment 100 of FIGS. 1-3. The main body 140 has a central main body portion 152 that connects first and second main body waist portions 148, 150. The first and second main body waist portions 148, 150 are connected on each lateral side of the garment by a side panel 260. Each side panel 260 preferably has a first side portion 266 attached to a lateral edge 168 of the main body 140 adjacent the first main body waist portion 148 and a portion of the central main body portion 152. Each side panel 260 also preferably has a second side portion 274 attached to the main body lateral edge 168 adjacent the second main body waist portion 150 and a portion of the central main body portion 152. The first and second side portions 266, 274 are joined at a side seam 230. The first and second side seam portions 266, 274 are joined and the side seam 230 is formed with the first and second side seam portions 266, 274 in a fully stretched condition. The side seams 230 of the garment 200 are formed in a generally straight line that may be substantially parallel to the vertical axis 202.

The first and second main body waist portions 148, 150 combine with the side panels 260 to form the waist opening edges 231 and the waist opening 232. The central main body portion 152 combines with the side panels 260 to form the leg opening edges 133 and the leg openings 234. The side panels 260 are preferably formed from an elastic or elasticized material to allow the waist opening 232 and the leg openings 234 to expand and contract to provide a secure, leak-free fit.

The garment 200 includes an adjustment arrangement configured to allow the adjustment of the side panels 260 to better conform to the body shape of the wearer. The adjustment arrangement preferably includes a pair of adjustment tabs 280 that include the respective side seams 230 on either side of the garment and that extend laterally outward from the inner edges 239 of the side seams 230. The adjustment tabs 280 are configured so that they can be grasped and pulled outward and then toward the first main body waist portion 148 (or the second main body waist portion 150) to adjust the fit of the garment 200.

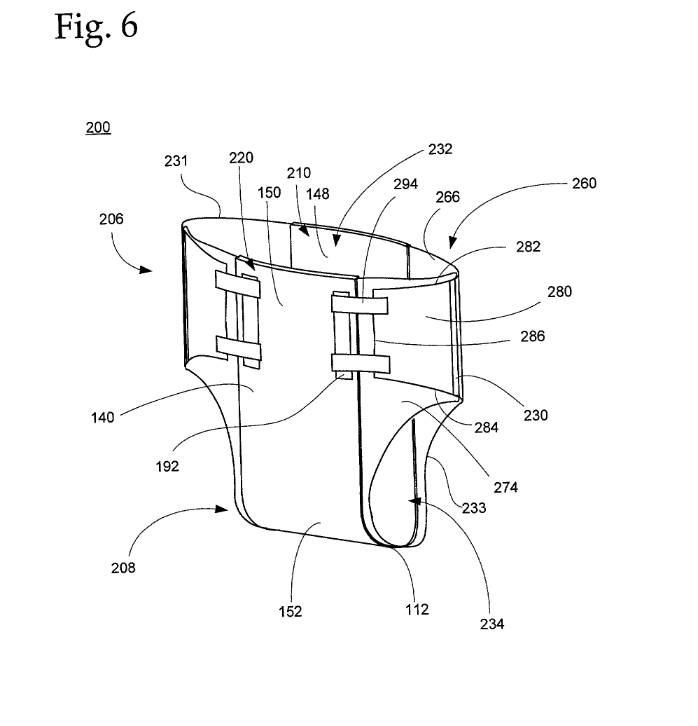

Each adjustment tab 280 may be removably connected to the main body 140 using a fastening arrangement that includes a pair of attachment strips 294. The attachment strips 294 are attached to the adjustment tab 280 so that they extend laterally outward as shown in FIGS. 5 and 7 and can be drawn toward the main body 140 as shown in FIG. 6. A gripping member 290 is attached to each attachment strip 294. The gripping member 290 is configured to releasably attach the attachment strip 294 to a receiving portion 192 on the main body 140. The gripping member 290 may comprise a hook surface such as the hook portion of a hook-and-loop fastener, the hook surface being adapted for reversible mating engagement with the receiving portion 192. In another embodiment, the gripping member 290 may comprise a releasable pressure sensitive adhesive for releasably adhering the attachment strip 294 to the receiving portion 192 of the main body 140. A protective release cover may be applied to the adhesive surface to protect the adhesive surface from contamination prior to use. The release cover may be removed just prior to use of the adjustment tabs 280 to adjust the fit of the garment 200.

The attachment strips 294 may comprise cloth, film, nonwoven material, or any other suitable material. In one embodiment, the attachment strips 294 comprise strips of nonwoven material having relatively little elastic extensibility and having a good tactile impression that is comfortable to the touch and will not irritate a user's skin. This may include a laminate of spun-bonded material and meltblown polypropylene or meltblown polyethylene. In another embodiment, the attachment strips 294 may comprise elastic material sandwiched between layers of nonwoven material, or other extensible materials, to provide for elastically extensible attachment strips 294.

Although the illustrated embodiment includes two attachment strips 294, any number may be used. One or more attachment strips 294 are preferably positioned adjacent the lateral tab edge 286 of the adjustment tab 280. An attachment strip 294 may be positioned adjacent the upper tab edge 282 for securing an adjustment of the waist opening 232. Alternatively or in addition, an attachment strip 294 may be positioned adjacent the lower tab edge 284 for securing an adjustment of one of the leg openings 234. In an alternative embodiment, a single wide attachment strip 294 may be disposed to extend from the lateral tab edge 286, the width of the attachment strip extending from the area adjacent the upper tab edge 282 to the area adjacent the lower tab edge 284.

The attachment strips 294 serve to secure the adjustment tabs 280 in place while maintaining tension on the side seams 230. In an embodiment having two attachment strips 294 extending from the corners of the adjustment tab 280, this may result in a fastened configuration such as that shown in FIG. 6. The fastened configuration of FIG. 6 has the effect of snugging the garment in place against the body of the wearer. It also has the effect of reducing the circumference of the waist opening 232 and associated leg opening 234. It will be understood that the use of the attachment strips 294 eliminates the need for the adjustment tabs 280 to be extendible as far as the main body. This reduces the material required to form the adjustment tabs 280.

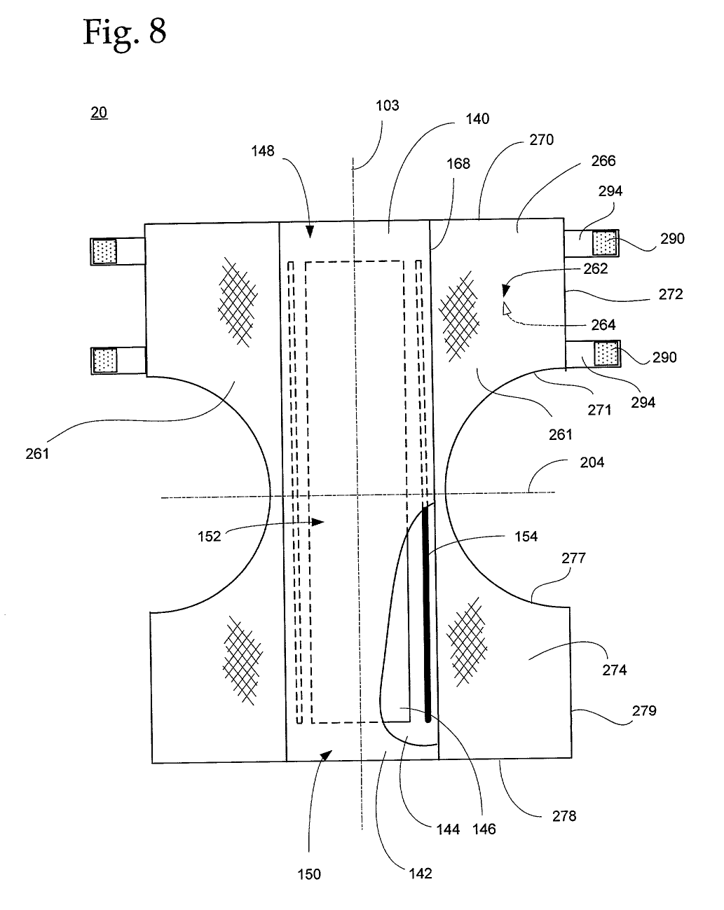

The components of the absorbent garment 200 are preferably assembled into a generally planar structure that can be doubled to form the U-shaped structure illustrated in FIGS. 5-7. FIG. 8 illustrates an unfinished garment 20 that can be used as a precursor to the absorbent final garment 200. At this intermediate stage of construction, the unfinished garment 20 is fully assembled except for the formation of the side seams. Several different final embodiments may be constructed from the unfinished garment 20, including the absorbent garment 200 of FIGS. 5-7.

FIG. 8 is a plan view of the body-facing side of the unfinished garment 20 in a generally flattened condition. The unfinished garment 20 may have a generally hourglass shaped structure, but it may also have any other shape suitable for the given application. The flattened unfinished garment 20 has a main body 140 that is substantially similar to that of the unfinished garment 10 of FIG. 4.

The unfinished garment 20 is provided with side panels 260 extending laterally outward from the lateral edges 168 of the main body 140. Each side panel 260 may be formed using a single continuous piece of elastic material having a body facing surface 262 and an opposing outward facing surface 264 as shown in FIG. 8. Alternatively, the side panel 260 may be formed from multiple portions of elastic material. For example, one portion of material could be used in the area adjacent the first main body waist portion 148 and a second portion of material could be used in the area adjacent the second main body waist portion 150. It will be understood that if multiple portions of elastic material are used to form the side panel 260, the side panel 260 could be formed without elastic material adjacent the central main body portion 152.

Each side panel 260 preferably has a first side portion 266 attached to a lateral edge 168 of the main body 140 adjacent the first main body waist portion 148 and a portion of the central main body portion 152. The first side portion 266 has a waist opening edge 270 and a lateral side panel edge 272. Each side panel 260 also has a second side portion 274 attached to the main body lateral edge 168 adjacent the second main body waist portion 150 and a portion of the central main body portion 152. The second side portion 274 has a waist opening edge 278 and a lateral side panel edge 279.

In some embodiments of the invention, the first and second side portions 266, 274 may be integrally formed with one or more components of the main body 140. In an exemplary preferred embodiment, the first and second side portions 266, 274 are formed by portions of the backsheet 144 that extend laterally outward beyond the lateral edges 168 of the main body 140.

The first and second side portions 266, 274 are configured so that the body facing surface 262 in the area adjacent the lateral side panel edge 272 of the first side portion 266 may be made to engage the body facing surface 262 in the area adjacent the lateral side panel edge 279 of the second side portion 274 when the garment 20 is folded to convert it from the configuration of FIG. 8 to a final assembled configuration. With the body facing surface 262 of the first and second side portions 266, 274 so-engaged and in a fully stretched condition, the first and second side portions 266, 274 are joined by the formation of side seam 230 thereon.

The formation of the side seam 230 produces a side panel from the first and second side portions 266, 274. By forming a straight, substantially vertical side seam 230 on each side panel 260 of the unfinished garment 20, a completed absorbent garment 200 is formed. As will be discussed, other garment embodiments may be formed from the unfinished garment 20 by forming side seams having a different geometry. The areas of the first and second side portions 266, 274 that are lateral to the side seam 130 may be joined in any suitable fashion to form the adjustment tabs 280. Additional materials may be added to these areas to enhance the strength of the adjustment tabs 280. In an alternative embodiment, one of the side portions may be cut adjacent the side seam 230, leaving the other side portion for use in forming the adjustment tab 280. In still another embodiment, the adjustment tab 280 may be formed by attaching a separate sheet or composite material to the side portions 266, 274 at or adjacent the side seam 230.

In embodiments where the first side portion 266 is used to form the adjustment tab 280, attachment strips 294 may be attached to the outward facing surface 264 of the first side portion 266. This is preferably accomplished before the side seam 230 is formed as shown in FIG. 8. It will be understood that if the second side portion 174 is used to form the adjustment tab 280 but the first side portion 266 is not, the attachment strips 294 may be attached to the body facing surface 262 of the second side portion 174. Although illustrated with two attachment strips 294 on each adjustment tab 280, it will be understood that the unfinished garment 20 may have any number of attachment strips 294.

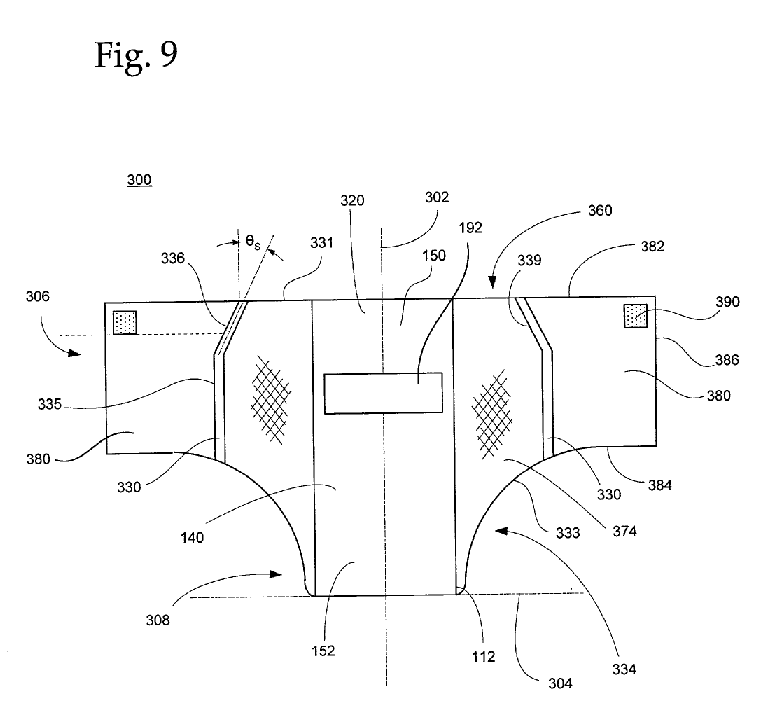

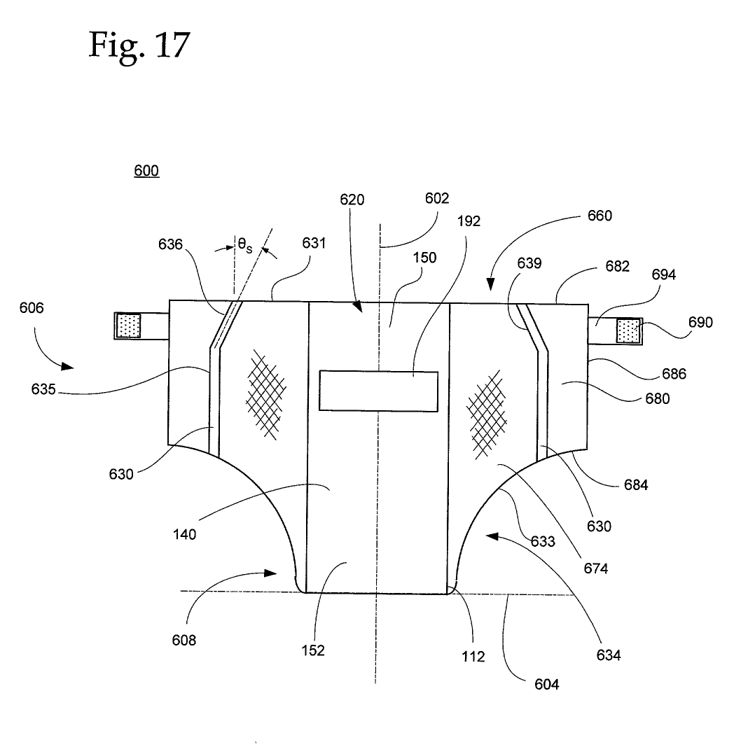

As previously noted, other absorbent garment embodiments of the present invention may be constructed from the unfinished garments 10 and 20 through the formation of side seams having different geometries. These embodiments allow the tailoring of absorbent garments to different body shapes and types. FIGS. 9 and 10, 11 and 12, 13 and 14, and 15 and 16 illustrate four embodiments of absorbent garments that may be formed from the unfinished garment 10 of FIG. 4. FIGS. 17 and 18, and 19 and 20 illustrate two embodiments of absorbent garments that may be formed from the unfinished garment 20 of FIG. 8.

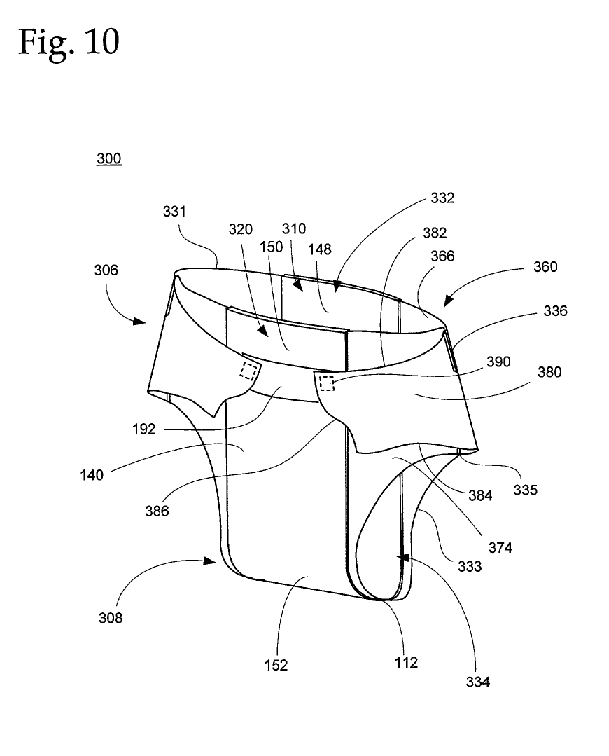

FIGS. 9 and 10 illustrate an absorbent garment 300 that may be formed from the unfinished garment 10 of FIG. 4. FIG. 9 is a front view of the garment 300 in a non-fastened configuration and FIG. 10 is a perspective view of the garment 300 in a fastened configuration. The absorbent garment 300 differs from the absorbent garment 100 of FIGS. 1-3 in that it has a side seam 330 that is formed from a straight, vertical portion 335 and an angled portion 336 that intersects the waist opening edge 331. This provides a waist opening 332 that may be somewhat smaller relative to the other proportions of the garment 300 as compared to the waist opening 132 of the absorbent garment 100. This configuration may be used, for example, to provide additional comfort and leakage security in wearers having disproportionately small thighs as compared to the wearer's waist.

FIG. 9 illustrates the garment 300 as it would appear when initially donned by the wearer. The garment 300 is shown with and will be described relative to first and second orthogonal axes 302, 304. The first axis 302 may be referred to as the vertical axis 302 and the second axis 304 may be referred to as the lateral axis 304. It will be understood by those having ordinary skill in the art that the vertical axis 302 corresponds generally to a head-to-toe axis of a standing wearer and that the lateral axis 304 corresponds generally to a side-to-side axis of a standing wearer. The lateral axis 304 further corresponds to the lateral axis 104 of the unfinished garment 10.

The absorbent garment 300 has a waist-encircling region 306 and a crotch region 308. The waist-encircling region may include a first waist portion 310, disposable adjacent to the back waist area of a wearer's body, and a second waist portion 320, disposable adjacent to the front waist area of a wearer's body. The first and second waist portions 310, 320 are joined to one another at side seams 330 to form the waist encircling portion 306 and to define a waist opening edge 331 surrounding a waist opening 332 and leg opening edges 333 surrounding two leg openings 334.

The absorbent garment 300 includes a main body 140 that is substantially similar to that of the absorbent garment 100 of FIGS. 1-3. The main body 140 has a central main body portion 152 that connects first and second main body waist portions 148, 150. The first and second main body waist portions 148, 150 are connected on each lateral side of the garment by a side panel 360. Each side panel 360 preferably has a first side portion 366 attached to a lateral edge 168 of the main body 140 adjacent the first main body waist portion 148 and a portion of the central main body portion 152. Each side panel 360 also preferably has a second side portion 374 attached to the main body lateral edge 168 adjacent the second main body waist portion 150 and a portion of the central main body portion 152. The first and second side portions 366, 374 are joined at the side seam 330. The first and second side seam portions 366, 374 are joined and the side seam 330 is formed with the first and second side seam portions 366, 374 in a fully stretched condition.

The first and second main body waist portions 148, 150 combine with the side panels 360 to form the waist opening edge 331 and the waist opening 332. The central main body portion 152 combines with the side panels 360 to form the leg opening edges 333 and the leg openings 334.

The side seam 330 is formed with a first straight portion 335 that intersects the leg opening edge 333 and a second straight portion 336 that intersects the waist opening edge 331. The first straight portion 335 is preferably substantially vertical (i.e., is within about 5 degrees of being parallel to the vertical axis 102). The second straight portion 336 defines a seam angle θ_{S} with respect to a line parallel to the vertical axis 102. The seam angle θ_{S}, which may be specifically tailored to accommodate different body types and sizes, may practicably be in a range of about 10 degrees to about 80 degrees. The seam angle θ_{S} is preferably in a range of about 10 degrees to about 50 degrees and most preferably in a range of about 25 degrees to about 35 degrees.

The garment 300 includes an adjustment arrangement that includes a pair of adjustment tabs 380 that include the respective side seams 330 on either side of the garment and that extend laterally outward from the inner edges 339 of the side seams 330. The adjustment tabs 380 are configured so that they can be grasped and pulled laterally outward and then toward the first main body waist portion 148 (or the second main body waist portion 150) to adjust the fit of the garment 300.

The angled side seam portion 336 allows the adjustment tab 380 to be used to separately adjust the fit of the waist encircling portion 306 of the garment 300. This may be accomplished by drawing the upper portion of the adjustment tab 380 across the side panel 360 and toward the main body 140 in a direction that is roughly perpendicular to the second straight portion 336 of the side seam 330. As shown in FIG. 10, this causes a fold in the second side portion 374 of the side panel 360 that is roughly parallel to the position of the second straight portion 336 of the side seam 330 prior to adjustment. This approach allows the waist opening 332 to be adjusted without significantly affecting the leg openings 334.

The adjustment arrangement includes a fastening arrangement configured for removably securing the adjustment tabs 380 to the main body 140. The fastening arrangement may include one or more gripping members 390 attached directly to the adjustment tab 380 as shown in FIGS. 9 and 10. The gripping members 390 are configured to releasably attach the adjustment tabs 380 to a receiving portion 192 on the main body 140. The gripping members 390 may each comprise a hook surface such as the hook portion of a hook-and-loop fastener, the hook surface being adapted for reversible mating engagement with the receiving portion 192. The receiving portion 192 may include a corresponding loop surface such as the loop portion of a hook-and-loop portion. The receiving portion 192 may be a single area to which both adjustment tabs 380 may be attached or may comprise a plurality of smaller areas. Alternatively, some or all of the backsheet used to form the main body 140 may be formed using a material that is grippable by the hook surface. In another embodiment, the gripping member 390 may comprise a releasable pressure sensitive adhesive for releasably adhering the adjustment tab 380 to the receiving portion 192 of the main body 140. A protective release cover may be applied to the adhesive surface to protect the adhesive surface from contamination prior to use. The release cover may be removed just prior to use of the adjustment tabs 380 to adjust the fit of the garment 300.

The fastening arrangement of the garment 300 preferably includes a gripping member 390 positioned adjacent the upper tab edge 382 and the lateral tab edge 386 of each adjustment tab 380. Additional gripping members 390 may be positioned adjacent the lateral tab edge 386 of the adjustment tab 380. In particular, a gripping member 390 may be positioned adjacent the lower tab edge 384 for separately adjusting the leg opening 334.

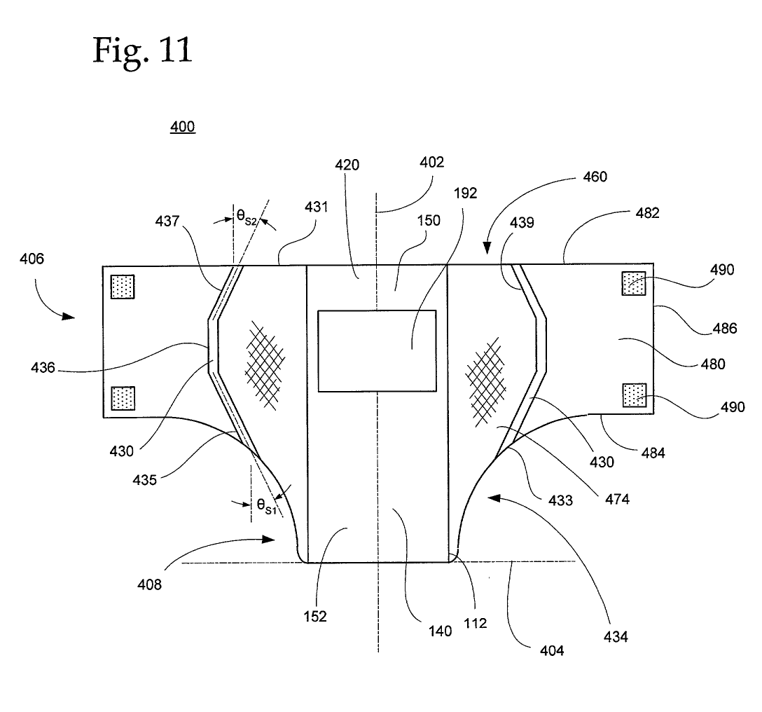

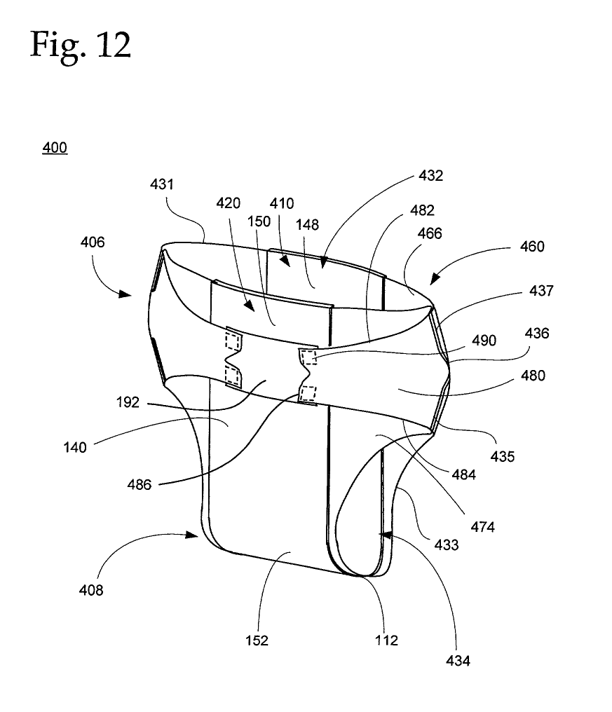

FIGS. 11 and 12 illustrate an absorbent garment 400 that may be formed from the unfinished garment 10. FIG. 11 is a front view of the garment 400 in a non-fastened configuration and FIG. 12 is a perspective view of the garment 400 in a fastened configuration. The absorbent garment 400 differs from the absorbent garment 300 of FIGS. 9 and 10 in that it has side seams 430 that are angled both at the waist opening and at the leg openings.

FIG. 11 illustrates the garment 400 as it would appear when initially donned by the wearer. The garment 400 is shown with and will be described relative to first and second orthogonal axes 402, 404. The first axis 402 may be referred to as the vertical axis 402 and the second axis 404 may be referred to as the lateral axis 404. It will be understood by those having ordinary skill in the art that the vertical axis 402 corresponds generally to a head-to-toe axis of a standing wearer and that the lateral axis 404 corresponds generally to a side-to-side axis of a standing wearer. The lateral axis 404 further corresponds to the lateral axis 104 of the unfinished garment 10.

The absorbent garment 400 has a waist-encircling region 406 and a crotch region 408. The waist-encircling region may include a first waist portion 410, disposable adjacent to the back waist area of a wearer's body, and a second waist portion 420, disposable adjacent to the front waist area of a wearer's body. The first and second waist portions 410, 420 are joined to one another at side seams 430 to form the waist encircling portion 406 and to define a waist opening edge 431 surrounding a waist opening 432 and leg opening edges 433 surrounding two leg openings 434.

The absorbent garment 400 includes a main body 140 that is substantially similar to that of the absorbent garment 100 of FIGS. 1-3. The main body 140 has a central main body portion 152 that connects first and second main body waist portions 148, 150. The first and second main body waist portions 148, 150 are connected on each lateral side of the garment by a side panel 460. Each side panel 460 preferably has a first side portion 466 attached to a lateral edge 168 of the main body 140 adjacent the first main body waist portion 148 and a portion of the central main body portion 152. Each side panel 460 also preferably has a second side portion 474 attached to the main body lateral edge 168 adjacent the second main body waist portion 150 and a portion of the central main body portion 152. The first and second side portions 466, 474 are joined at the side seam 430. The first and second side seam portions 466, 474 are joined and the side seam 430 is formed with the first and second side seam portions 466, 474 in a fully stretched condition.

The first and second main body waist portions 148, 150 combine with the side panels 460 to form the waist opening edge 431 and the waist opening 432. The central main body portion 152 combines with the side panels 460 to form the leg opening edges 433 and the leg openings 434.

The side seam 430 is formed with a first straight portion 435 that intersects the leg opening edge 433, a second straight portion 437 that intersects the waist opening edge 431 and a third straight portion 436 that connects the first and second straight portions 435, 436. The third straight portion 436 is preferably substantially vertical (i.e., is within about 5 degrees of being parallel to the vertical axis 102). The first straight portion 435 angles inward toward the main body 140 and defines a first seam angle θ_{S1} with respect to a line parallel to the vertical axis 102. The first seam angle θ_{S1}, which may be specifically tailored to accommodate different body types and sizes, may practicably be in a range of about 10 degrees to about 80 degrees. The first seam angle θ_{S1} is preferably in a range of about 10 degrees to about 50 degrees and most preferably in a range of about 25 degrees to about 35 degrees. The second straight portion 437 also angles inward toward the main body and defines a second seam angle θ_{S2} with respect to a line parallel to the vertical axis 102. The second seam angle θ_{S2} may also be tailored to accommodate different body types and sizes and may practicably be in a range of about 10 degrees to about 80 degrees. The second seam angle θ_{S2} is preferably in a range of about 10 degrees to about 50 degrees and most preferably in a range of about 25 degrees to about 35 degrees. The side seam 430 may optionally be configured so that the first and second seam angles θ_{S1}, θ_{S2} may be substantially equal.

The garment 400 includes an adjustment arrangement that includes a pair of adjustment tabs 480 that include the respective side seams 430 on either side of the garment and that extend laterally outward from the inner edges 439 of the side seams 430. The adjustment tabs 480 are configured so that they can be grasped and pulled laterally outward and then toward the first main body waist portion 148 (or the second main body waist portion 150) to adjust the fit of the garment 400.

The angled portions 435, 437 of the side seam 430 allow the adjustment tab 480 to be used to separately adjust the fit of the waist encircling portion 406 and each of the leg openings 434. This may be accomplished by drawing the lower portion of the adjustment tab 480 across the side panel 460 and toward the main body 140 in a direction that is roughly perpendicular to the first straight portion 435 and by drawing an upper portion of the adjustment tab 480 across the side panel 460 and toward the main body 140 in a direction that is roughly perpendicular to the second straight portion 436. As shown in FIG. 12, this causes a first fold in the second side panel portion 474 that is roughly parallel to the position of the first side seam portion 435 prior to adjustment and a second fold in the second side panel portion 774 that is roughly parallel to the position of the second side seam portion 437 prior to adjustment. This approach allows the waist opening 432 to be adjusted without significantly affecting the leg openings 434 and vice versa.

The adjustment arrangement includes a fastening arrangement configured for removably securing the adjustment tabs 480 to the main body 140. The fastening arrangement may include one or more gripping members 490 attached directly to the adjustment tab 480 as shown in FIGS. 11 and 12. The gripping members 490 are configured to releasably attach the adjustment tabs 480 to a receiving portion 192 on the main body 140. The gripping members 490 may each comprise a hook surface such as the hook portion of a hook-and-loop fastener, the hook surface being adapted for reversible mating engagement with the receiving portion 192. The receiving portion 192 may include a corresponding loop surface such as the loop portion of a hook-and-loop portion. The receiving portion 192 may be a single area to which both adjustment tabs 480 may be attached or may comprise a plurality of smaller areas. Alternatively, some or all of the backsheet used to form the main body 140 may be formed using a material that is grippable by the hook surface. In another embodiment, the gripping member 490 may comprise a releasable pressure sensitive adhesive for releasably adhering the adjustment tab 480 to the receiving portion 192 of the main body 140. A protective release cover may be applied to the adhesive surface to protect the adhesive surface from contamination prior to use. The release cover may be removed just prior to use of the adjustment tabs 480 to adjust the fit of the garment 400.

The fastening arrangement of the garment 400 preferably includes a gripping member 490 positioned adjacent the upper tab edge 482 and the lateral tab edge 486 of each adjustment tab 480 for fastening the upper portion of the adjustment tab 480 to the main body 140. The fastening arrangement of the garment 400 also preferably includes a gripping member 490 positioned adjacent the lower tab edge 482 and the lateral tab edge 486 of each adjustment tab 480 for fastening the lower portion of the adjustment tab 480 to the main body 140. Additional gripping members 490 may also be positioned adjacent the lateral tab edge 486 of the adjustment tab 480.

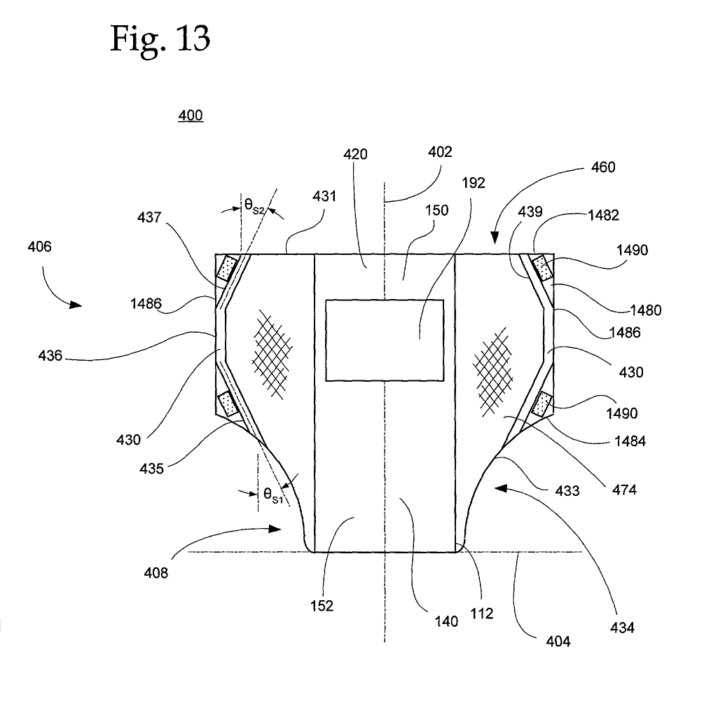

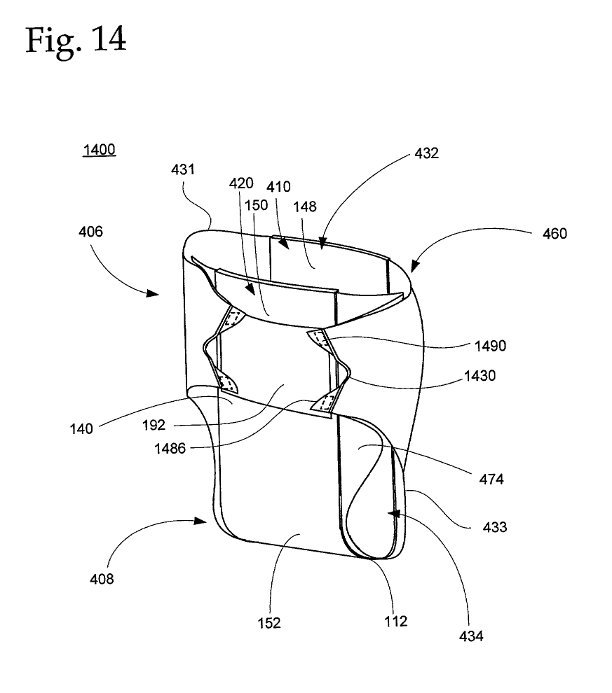

A variation of the previous garment is shown in FIGS. 13 and 14. The garment 1400 of FIGS. 13 and 14 is substantially similar to the garment 400 of FIGS. 11 and 12 except that the adjustment tabs 1480 have a lateral tab edge that is coextensive with the outer edge of the third straight portion 436 of the side seam 430. This significantly shortens the upper and lower tab edges 482, 484 and reduces the amount of material required for the adjustment tabs 1480. As shown, the gripping members 1490 used in this embodiment may be rectangular and positioned so as to be substantially parallel to the angled side seam portions 435, 437.

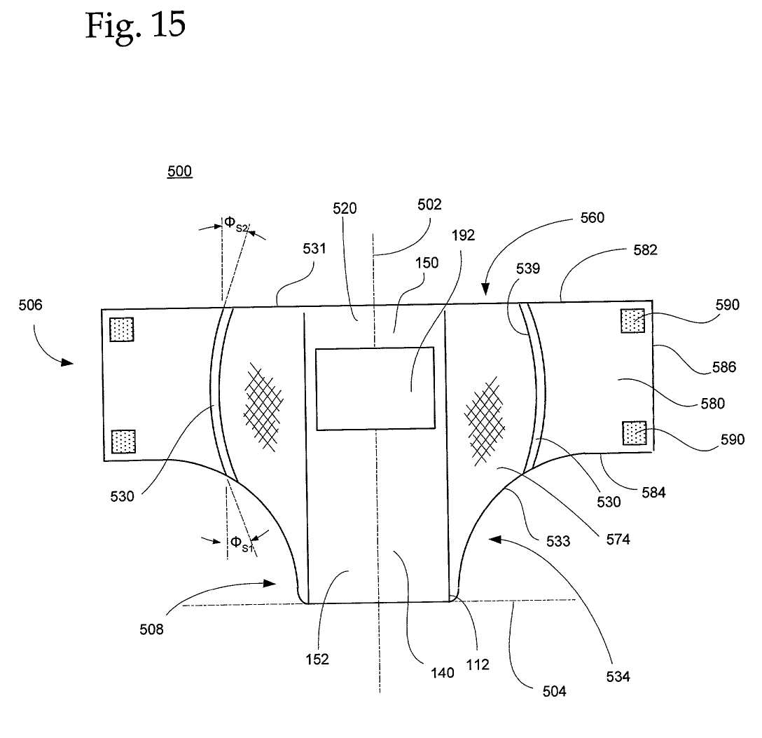

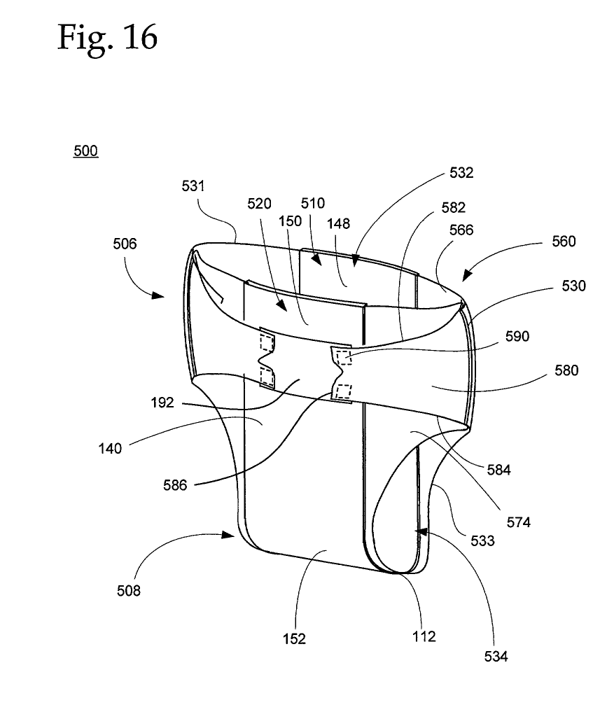

FIGS. 15 and 16 illustrate an absorbent garment 500 that may be formed from the unfinished garment 10. FIG. 15 is a front view of the garment 500 in a non-fastened configuration and FIG. 16 is a perspective view of the garment 500 in a fastened configuration. The absorbent garment 500 differs from the absorbent garment 100 of FIGS. 1-3 in that it has side seams 530 that are curved and that may intersect the waist opening and the leg openings of the garment at an angle. The use of a curved side seam 530 provides significant advantages in comfort, security and appearance as detailed in co-pending application Ser. No. 10/074,028 (Attorney Docket No. 53394.000566).

FIG. 15 illustrates the garment 500 as it would appear when initially donned by the wearer. The garment 500 is shown with and will be described relative to first and second orthogonal axes 502, 504. The first axis 502 may be referred to as the vertical axis 502 and the second axis 504 may be referred to as the lateral axis 504. It will be understood by those having ordinary skill in the art that the vertical axis 502 corresponds generally to a head-to-toe axis of a standing wearer and that the lateral axis 504 corresponds generally to a side-to-side axis of a standing wearer. The lateral axis 504 further corresponds to the lateral axis 104 of the unfinished garment 10.

The absorbent garment 500 has a waist-encircling region 506 and a crotch region 508. The waist-encircling region may include a first waist portion 510, disposable adjacent to the back waist area of a wearer's body, and a second waist portion 520, disposable adjacent to the front waist area of a wearer's body. The first and second waist portions 510, 520 are joined to one another at side seams 530 to form the waist encircling portion 506 and to define a waist opening edge 531 surrounding a waist opening 532 and leg opening edges 533 surrounding two leg openings 534.

The absorbent garment 500 includes a main body 140 that is substantially similar to that of the absorbent garment 100 of FIGS. 1-3. The main body 140 has a central main body portion 152 that connects first and second main body waist portions 148, 150. The first and second main body waist portions 148, 150 are connected on each lateral side of the garment by a side panel 560. Each side panel 560 preferably has a first side portion 566 attached to a lateral edge 168 of the main body 140 adjacent the first main body waist portion 148 and a portion of the central main body portion 152. Each side panel 560 also preferably has a second side portion 574 attached to the main body lateral edge 168 adjacent the second main body waist portion 150 and a portion of the central main body portion 152. The first and second side portions 566, 574 are joined at the side seam 530. The first and second side seam portions 566, 574 are joined and the side seam 530 is formed with the first and second side seam portions 566, 574 in a fully stretched condition.

The first and second main body waist portions 148, 150 combine with the side panels 560 to form the waist opening edge 531 and the waist opening 532. The central main body portion 152 combines with the side panels 560 to form the leg opening edges 533 and the leg openings 534.

The side seam 530 is formed so that at least a portion of the side seam 530 is curved. In the illustrated embodiment, the side seam 530 is formed as a circular arc with a constant radius. It will be understood that other configurations are possible in which the side seam 530 is a composite of a plurality of curves having any predetermined geometric or freeform shape. The geometry of the side seam 530 may also be a composite of curved and straight portions.

Regardless of the specific geometry of the side seam 530, the side seam 530 defines a first seam angle Φ_{S1} with respect to a line parallel to the vertical axis 102 at the point where the side seam 530 intersects the leg opening edge 533. The side seam 530 defines a second seam angle Φ_{S2} with respect to a line parallel to the vertical axis 102 at the point where the side seam 530 intersects the waist opening edge 531. As shown in FIG. 15, if the side seam 530 is curved at the intersection points the seam angles are defined by the tangent to the curve at the intersection point. The first seam angle Φ_{S1}, which may be specifically tailored to accommodate different body types and sizes, may practicably be in a range of about 10 degrees to about 80 degrees. The first seam angle Φ_{S1} is preferably in a range of about 10 degrees to about 50 degrees and most preferably in a range of about 25 degrees to about 35 degrees. The second seam angle Φ_{S2} is preferably in a range of about 10 degrees to about 50 degrees and most preferably in a range of about 25 degrees to about 35 degrees. The side seam 530 may optionally be configured so that the first and second seam angles Φ_{S1}, Φ_{S2} may be substantially equal.

The garment 500 includes an adjustment arrangement that includes a pair of adjustment tabs 580 that include the respective side seams 530 on either side of the garment and that extend laterally outward from the inner edges 539 of the side seams 530. The adjustment tabs 580 are configured so that they can be grasped and pulled laterally outward and then toward the first main body waist portion 148 (or the second main body waist portion 150) to adjust the fit of the garment 500.

The adjustment tab 580 may be used to separately adjust the fit of the waist encircling portion 506 and each of the leg openings 534. This may be accomplished by drawing the upper portion of the adjustment tab 580 across the side panel 560 and toward the main body 140 in a direction that is roughly perpendicular to the tangent to the curved seam 530 at its intersection with the waist opening edge 531 and drawing a lower portion of the second side panel portion 574 across the side panel 560 and toward the main body 140 in a direction that is roughly perpendicular to the tangent to the curved seam 530 at its intersection with the leg opening edge 533. This approach allows the waist opening 532 to be adjusted without significantly affecting the leg openings 534 and vice versa.

The adjustment arrangement includes a fastening arrangement configured for removably securing the adjustment tabs 580 to the main body 140. The fastening arrangement may include one or more gripping members 590 attached directly to the adjustment tab 580 as shown in FIGS. 15 and 16. The gripping members 590 are configured to releasably attach the adjustment tabs 580 to a receiving portion 192 on the main body 140. The gripping members 590 may each comprise a hook surface such as the hook portion of a hook-and-loop fastener, the hook surface being adapted for reversible mating engagement with the receiving portion 192. The receiving portion 192 may include a corresponding loop surface such as the loop portion of a hook-and-loop portion. The receiving portion 192 may be a single area to which both adjustment tabs 580 may be attached or may comprise a plurality of smaller areas. Alternatively, some or all of the backsheet used to form the main body 140 may be formed using a material that is grippable by the hook surface. In another embodiment, the gripping member 590 may comprise a releasable pressure sensitive adhesive for releasably adhering the adjustment tab 580 to the receiving portion 192 of the main body 140. A protective release cover may be applied to the adhesive surface to protect the adhesive surface from contamination prior to use. The release cover may be removed just prior to use of the adjustment tabs 580 to adjust the fit of the garment 500.

The fastening arrangement of the garment 500 preferably includes a gripping member 590 positioned adjacent the upper tab edge 582 and the lateral tab edge 586 of each adjustment tab 580 for fastening the upper portion of the adjustment tab 580 to the main body 140. The fastening arrangement of the garment 500 also preferably includes a gripping member 590 positioned adjacent the lower tab edge 582 and the lateral tab edge 586 of each adjustment tab 580 for fastening the lower portion of the adjustment tab 580 to the main body 140. Additional gripping members 590 may also be positioned adjacent the lateral tab edge 586 of the adjustment tab 580.

It will be understood that other absorbent garment embodiments may be constructed from the unfinished absorbent garment 10, including, for example, embodiments having side seams with a straight portion adjacent the waist opening and an angled portion adjacent the leg opening and embodiments that have a substantially vertical straight portion adjacent the waist opening and a curved portion adjacent the leg opening or vice versa.

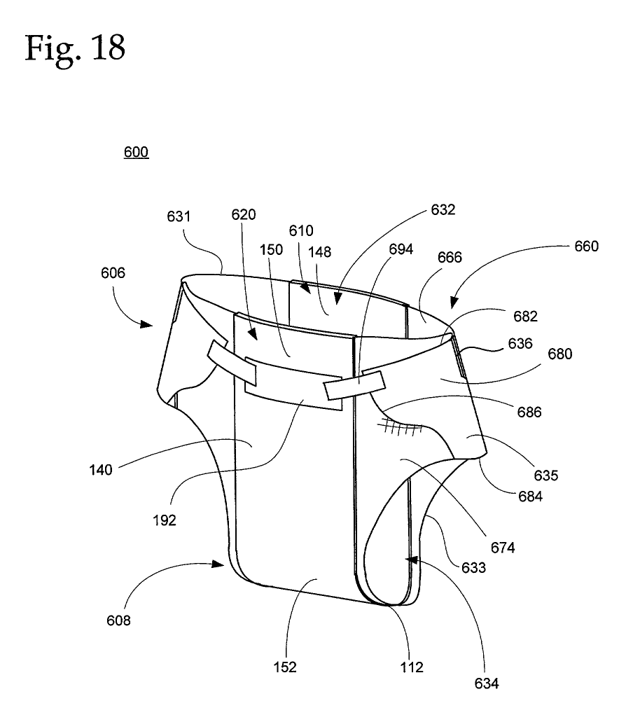

FIGS. 17 and 18 illustrate an absorbent garment 600 that may be formed from the unfinished garment 20. FIG. 17 is a front view of the garment 600 in a non-fastened configuration and FIG. 18 is a perspective view of the garment 600 in a fastened configuration. The absorbent garment 600 differs from the absorbent garment 200 of FIGS. 4-6 in that it has a side seam 630 that is formed from a straight, vertical portion 635 and an angled portion 636 that intersects the waist opening edge 631. This provides a waist opening 632 that may be somewhat smaller relative to the other proportions of the garment 600 as compared to the waist opening 132 of the absorbent garment 100. This configuration may be used, for example, to provide additional comfort and leakage security in wearers having disproportionately small thighs as compared to the wearer's waist.

FIG. 17 illustrates the garment 600 as it would appear when initially donned by the wearer. The garment 600 is shown with and will be described relative to first and second orthogonal axes 602, 604. The first axis 602 may be referred to as the vertical axis 602 and the second axis 604 may be referred to as the lateral axis 604. It will be understood by those having ordinary skill in the art that the vertical axis 602 corresponds generally to a head-to-toe axis of a standing wearer and that the lateral axis 604 corresponds generally to a side-to-side axis of a standing wearer. The lateral axis 604 further corresponds to the lateral axis 204 of the unfinished garment 20.

The absorbent garment 600 has a waist-encircling region 606 and a crotch region 608. The waist-encircling region may include a first waist portion 610, disposable adjacent to the back waist area of a wearer's body, and a second waist portion 620, disposable adjacent to the front waist area of a wearer's body. The first and second waist portions 610, 620 are joined to one another at side seams 630 to form the waist encircling portion 606 and to define a waist opening edge 631 surrounding a waist opening 632 and leg opening edges 633 surrounding two leg openings 634.

The absorbent garment 600 includes a main body 140 that is substantially similar to that of the absorbent garment 100 of FIGS. 1-3. The main body 140 has a central main body portion 152 that connects first and second main body waist portions 148, 150. The first and second main body waist portions 148, 150 are connected on each lateral side of the garment by a side panel 660. Each side panel 660 preferably has a first side portion 666 attached to a lateral edge 168 of the main body 140 adjacent the first main body waist portion 148 and a portion of the central main body portion 152. Each side panel 660 also preferably has a second side portion 674 attached to the main body lateral edge 168 adjacent the second main body waist portion 150 and a portion of the central main body portion 152. The first and second side portions 666, 674 are joined at the side seam 630. The first and second side seam portions 666, 674 are joined and the side seam 630 is formed with the first and second side seam portions 666, 674 in a fully stretched condition.

The first and second main body waist portions 148, 150 combine with the side panels 660 to form the waist opening edge 631 and the waist opening 632. The central main body portion 152 combines with the side panels 660 to form the leg opening edges 633 and the leg openings 634.

The side seam 630 is formed with a first straight portion 635 that intersects the leg opening edge 633 and a second straight portion 636 that intersects the waist opening edge 631. The first straight portion 635 is preferably substantially vertical (i.e., is within about 5 degrees of being parallel to the vertical axis 102). The second straight portion 636 defines a seam angle θ_{S} with respect to a line parallel to the vertical axis 102. The seam angle θ_{S}, which may be specifically tailored to accommodate different body types and sizes, may practicably be in a range of about 10 degrees to about 80 degrees. The seam angle θ_{S} is preferably in a range of about 10 degrees to about 50 degrees and most preferably in a range of about 25 degrees to about 35 degrees.

The garment 600 includes an adjustment arrangement that includes a pair of adjustment tabs 680 that include the respective side seams 630 on either side of the garment and that extend laterally outward from the inner edges 639 of the side seams 630. The adjustment tabs 680 are configured so that they can be grasped and pulled laterally outward and then toward the first main body waist portion 148 (or the second main body waist portion 150) to adjust the fit of the garment 600.

The angled side seam portion 636 allows the adjustment tab 680 to be used to separately adjust the fit of the waist encircling portion 606 of the garment 600. This may be accomplished by drawing the upper portion of the adjustment tab 680 across the side panel 660 and toward the main body 140 in a direction that is roughly perpendicular to the second straight portion 636 of the side seam 630. As shown in FIG. 18, this causes a fold in the second side portion 674 of the side panel 660 that is roughly parallel to the position of the second straight portion 636 of the side seam 630 prior to adjustment. This approach allows the waist opening 632 to be adjusted without significantly affecting the leg openings 634.

Each adjustment tab 680 may be removably connected to the main body 140 using a fastening arrangement that includes an attachment strip 694. The attachment strip 694 is attached to the adjustment tab 180 so that it extends laterally outward as shown in FIG. 17 and can be drawn toward the main body 140 as shown in FIG. 18. A gripping member 690 is attached to each attachment strip 694. The gripping member 690 is configured to releasably attach the attachment strip 694 to a receiving portion 192 on the main body 140. The gripping member 190 may comprise a hook surface such as the hook portion of a hook-and-loop fastener, the hook surface being adapted for reversible mating engagement with the receiving portion 192. In another embodiment, the gripping member 690 may comprise a releasable pressure sensitive adhesive for releasably adhering the attachment strip 694 to the receiving portion 192 of the main body 140. A protective release cover may be applied to the adhesive surface to protect the adhesive surface from contamination prior to use. The release cover may be removed just prior to use of the adjustment tabs 680 to adjust the fit of the garment 600.

As previously described, the attachment strips used in conjunction with the present invention may comprise cloth, film, nonwoven material, or any other suitable material. Although the illustrated embodiment includes one attachment strip 694 on each adjustment tab 680, any number of attachment strips 694 may be used. One or more attachment strips 694 are preferably positioned adjacent the upper tab edge 682 as shown in FIG. 17. An additional attachment strip 694 may be positioned adjacent the lower tab edge 684 for separately securing an adjustment of one of the leg openings 634.

The attachment strips 694 serve to secure the adjustment tabs 680 in place while maintaining tension on the side seams 630. The fastened configuration of FIG. 18 illustrates that securing the adjustment tabs 680 in this fashion has the effect of snugging the garment 600 in place against the body of the wearer. It also has the effect of reducing the circumference of the waist opening 632 and associated leg opening 634. It will be understood that the use of the attachment strips 694 eliminates the need for the adjustment tabs 680 to be extendible as far as the main body. This reduces the material required to form the adjustment tabs 680.

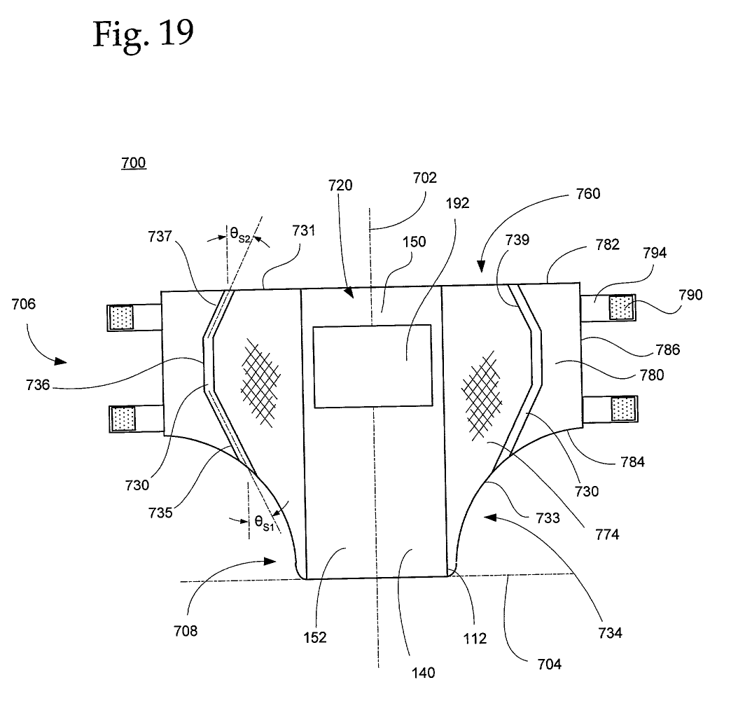

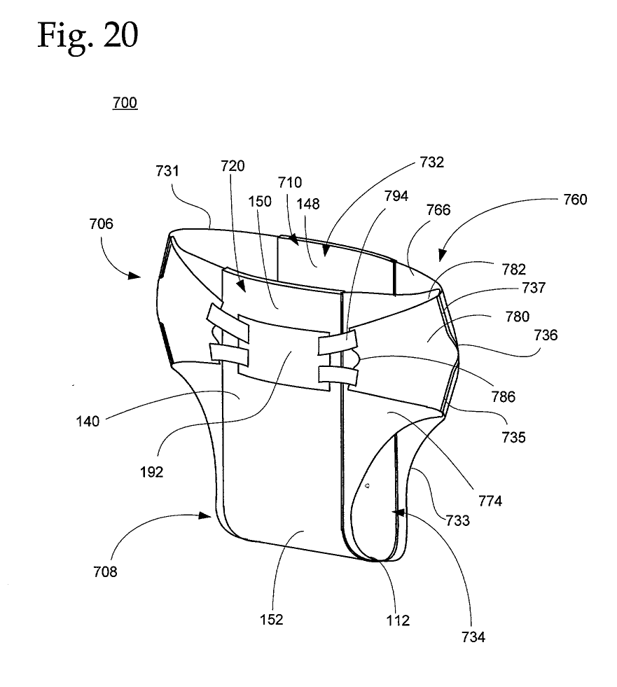

FIGS. 19 and 20 illustrate an absorbent garment 700 that may be formed from the unfinished garment 20. FIG. 19 is a front view of the garment 700 in a non-fastened configuration and FIG. 20 is a perspective view of the garment 700 in a fastened configuration. The absorbent garment 700 differs from the absorbent garment 600 of FIGS. 15 and 16 in that it has side seams 730 that are angled both at the waist opening and at the leg openings.

FIG. 19 illustrates the garment 700 as it would appear when initially donned by the wearer. The garment 700 is shown with and will be described relative to first and second orthogonal axes 702, 704. The first axis 702 may be referred to as the vertical axis 702 and the second axis 704 may be referred to as the lateral axis 704. It will be understood by those having ordinary skill in the art that the vertical axis 702 corresponds generally to a head-to-toe axis of a standing wearer and that the lateral axis 704 corresponds generally to a side-to-side axis of a standing wearer. The lateral axis 704 further corresponds to the lateral axis 204 of the unfinished garment 20.

The absorbent garment 700 has a waist-encircling region 706 and a crotch region 708. The waist-encircling region may include a first waist portion 710, disposable adjacent to the back waist area of a wearer's body, and a second waist portion 720, disposable adjacent to the front waist area of a wearer's body. The first and second waist portions 710, 720 are joined to one another at side seams 730 to form the waist encircling portion 706 and to define a waist opening edge 731 surrounding a waist opening 732 and leg opening edges 733 surrounding two leg openings 734.

The absorbent garment 700 includes a main body 140 that is substantially similar to that of the absorbent garment 100 of FIGS. 1-3. The main body 140 has a central main body portion 152 that connects first and second main body waist portions 148, 150. The first and second main body waist portions 148, 150 are connected on each lateral side of the garment by a side panel 760. Each side panel 760 preferably has a first side portion 766 attached to a lateral edge 168 of the main body 140 adjacent the first main body waist portion 148 and a portion of the central main body portion 152. Each side panel 760 also preferably has a second side portion 774 attached to the main body lateral edge 168 adjacent the second main body waist portion 150 and a portion of the central main body portion 152. The first and second side portions 766, 774 are joined at the side seam 730. The first and second side seam portions 766, 774 are joined and the side seam 730 is formed with the first and second side seam portions 766, 774 in a fully stretched condition.

The first and second main body waist portions 148, 150 combine with the side panels 760 to form the waist opening edge 731 and the waist opening 732. The central main body portion 152 combines with the side panels 760 to form the leg opening edges 733 and the leg openings 734.

The side seam 730 is formed with a first straight portion 735 that intersects the leg opening edge 733, a second straight portion 737 that intersects the waist opening edge 731 and a third straight portion 736 that connects the first and second straight portions 735, 737. The third straight portion 736 is preferably substantially vertical (i.e., is within about 5 degrees of being parallel to the vertical axis 102). The first straight portion 735 angles inward toward the main body 140 and defines a first seam angle θ_{S1} with respect to a line parallel to the vertical axis 102. The first seam angle θ_{S1}, which may be specifically tailored to accommodate different body types and sizes, may practicably be in a range of about 10 degrees to about 80 degrees. The first seam angle θ_{S1} is preferably in a range of about 10 degrees to about 50 degrees and most preferably in a range of about 25 degrees to about 35 degrees. The second straight portion 737 also angles inward toward the main body and defines a second seam angle θ_{S2} with respect to a line parallel to the vertical axis 102. The second seam angle θ_{S2} may also be tailored to accommodate different body types and sizes and may practicably be in a range of about 10 degrees to about 80 degrees. The second seam angle θ_{S2} is preferably in a range of about 10 degrees to about 50 degrees and most preferably in a range of about 25 degrees to about 35 degrees. The side seam 730 may optionally be configured so that the first and second seam angles θ_{S1}, θ_{S2} may be substantially equal.

The garment 700 includes an adjustment arrangement that includes a pair of adjustment tabs 780 that include the respective side seams 730 on either side of the garment and that extend laterally outward from the inner edges 739 of the side seams 730. The adjustment tabs 780 are configured so that they can be grasped and pulled laterally outward and then toward the first main body waist portion 148 (or the second main body waist portion 150) to adjust the fit of the garment 700.

The angled portions 735, 737 of the side seam 730 allow the adjustment tab 780 to be used to separately adjust the fit of the waist encircling portion 706 and each of the leg openings 734. This may be accomplished by drawing the lower portion of the adjustment tab 780 across the side panel 760 and toward the main body 140 in a direction that is roughly perpendicular to the first straight portion 735 and by drawing an upper portion of the adjustment tab 780 across the side panel 760 and toward the main body 140 in a direction that is roughly perpendicular to the second straight portion 736. As shown in FIG. 20, this causes a first fold in the second side panel portion 774 that is roughly parallel to the position of the first side seam portion 735 prior to adjustment and a second fold in the second side panel portion 774 that is roughly parallel to the position of the second side seam portion 437 prior to adjustment. This approach allows the waist opening 732 to be adjusted without significantly affecting the leg openings 734 and vice versa.

Each adjustment tab 780 may be removably connected to the main body 140 using a fastening arrangement that includes a pair of attachment strips 794. The attachment strips 794 are attached to the adjustment tab 780 so that they extend laterally outward as shown in FIG. 19 and can be drawn toward the main body 140 as shown in FIG. 20. A gripping member 790 is attached to each attachment strip 794. The gripping member 790 is configured to releasably attach the attachment strip 794 to a receiving portion 192 on the main body 140. The gripping member 790 may comprise a hook surface such as the hook portion of a hook-and-loop fastener, the hook surface being adapted for reversible mating engagement with the receiving portion 192. In another embodiment, the gripping member 790 may comprise a releasable pressure sensitive adhesive for releasably adhering the attachment strip 794 to the receiving portion 192 of the main body 140. A protective release cover may be applied to the adhesive surface to protect the adhesive surface from contamination prior to use. The release cover may be removed just prior to use of the adjustment tabs 780 to adjust the fit of the garment 700.

As previously described, the attachment strips used in conjunction with the present invention may comprise cloth, film, nonwoven material, or any other suitable material. Although the illustrated embodiment includes two attachment strips 794, any number may be used. One or more attachment strips 794 are preferably positioned adjacent the lateral tab edge 786 of the adjustment tab 780. An attachment strip 794 may be positioned adjacent the upper tab edge 782 for securing an adjustment of the waist opening 732. Alternatively or in addition, an attachment strip 794 may be positioned adjacent the lower tab edge 784 for securing an adjustment of one of the leg openings 734.

The attachment strips 794 serve to secure the adjustment tabs 780 in place while maintaining tension on the side seams 730. In an embodiment having two attachment strips 794 extending from the corners of the adjustment tab 780, this may result in a fastened configuration such as that shown in FIG. 20. The fastened configuration of FIG. 20 has the effect of snugging the garment in place against the body of the wearer. It also has the effect of reducing the circumference of the waist opening 732 and associated leg opening 734. It will be understood that the use of the attachment strips 794 eliminates the need for the adjustment tabs 780 to be extendible as far as the main body. This reduces the material required to form the adjustment tabs 780.

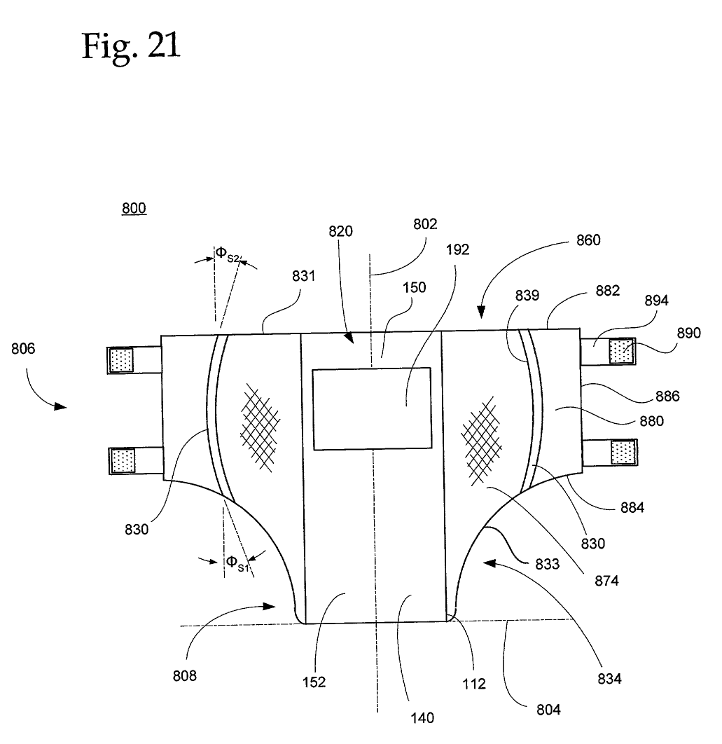

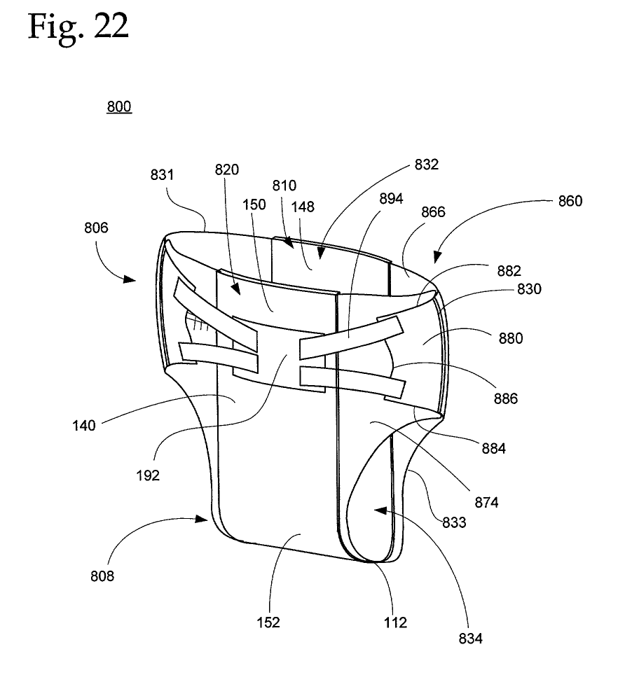

FIGS. 21 and 22 illustrate an absorbent garment 800 that may be formed from the unfinished garment 22. FIG. 21 is a front view of the garment 800 in a non-fastened configuration and FIG. 20 is a perspective view of the garment 800 in a fastened configuration. The absorbent garment 800 differs from the absorbent garment 200 of FIGS. 4-7 in that it has side seams 830 that are curved and that may intersect the waist opening and the leg openings of the garment at an angle. As previously noted, the use of a curved side seam 830 provides significant advantages in comfort, security and appearance.

FIG. 21 illustrates the garment 800 as it would appear when initially donned by the wearer. The garment 800 is shown with and will be described relative to first and second orthogonal axes 802, 804. The first axis 802 may be referred to as the vertical axis 802 and the second axis 804 may be referred to as the lateral axis 804. It will be understood by those having ordinary skill in the art that the vertical axis 802 corresponds generally to a head-to-toe axis of a standing wearer and that the lateral axis 804 corresponds generally to a side-to-side axis of a standing wearer. The lateral axis 804 further corresponds to the lateral axis 204 of the unfinished garment 20.

The absorbent garment 800 has a waist-encircling region 806 and a crotch region 808. The waist-encircling region may include a first waist portion 810, disposable adjacent to the back waist area of a wearer's body, and a second waist portion 820, disposable adjacent to the front waist area of a wearer's body. The first and second waist portions 810, 820 are joined to one another at side seams 830 to form the waist encircling portion 806 and to define a waist opening edge 831 surrounding a waist opening 832 and leg opening edges 833 surrounding two leg openings 834.

The absorbent garment 800 includes a main body 140 that is substantially similar to that of the absorbent garment 200 of FIGS. 4-7. The main body 140 has a central main body portion 152 that connects first and second main body waist portions 148, 150. The first and second main body waist portions 148, 150 are connected on each lateral side of the garment by a side panel 860. Each side panel 860 preferably has a first side portion 866 attached to a lateral edge 168 of the main body 140 adjacent the first main body waist portion 148 and a portion of the central main body portion 152. Each side panel 860 also preferably has a second side portion 874 attached to the main body lateral edge 168 adjacent the second main body waist portion 150 and a portion of the central main body portion 152. The first and second side portions 866, 874 are joined at the side seam 830. The first and second side seam portions 866, 874 are joined and the side seam 830 is formed with the first and second side seam portions 866, 874 in a fully stretched condition.

The first and second main body waist portions 148, 150 combine with the side panels 860 to form the waist opening edge 831 and the waist opening 832. The central main body portion 152 combines with the side panels 860 to form the leg opening edges 833 and the leg openings 834.

The side seam 830 is formed so that at least a portion of the side seam 830 is curved. In the illustrated embodiment, the side seam 830 is formed as a circular arc with a constant radius. It will be understood that other configurations are possible in which the side seam 830 is a composite of a plurality of curves having any predetermined geometric or freeform shape. The geometry of the side seam 830 may also be a composite of curved and straight portions.

Regardless of the specific geometry of the side seam 830, the side seam 830 defines a first seam angle Φ_{S1} with respect to a line parallel to the vertical axis 102 at the point where the side seam 830 intersects the leg opening edge 833. The side seam 830 defines a second seam angle Φ_{S2} with respect to a line parallel to the vertical axis 102 at the point where the side seam 830 intersects the waist opening edge 831. As shown in FIG. 21, if the side seam 830 is curved at the intersection points the seam angles are defined by the tangent to the curve at the intersection point. The first seam angle Φ_{S1}, which may be specifically tailored to accommodate different body types and sizes, may practicably be in a range of about 10 degrees to about 80 degrees. The first seam angle Φ_{S1} is preferably in a range of about 10 degrees to about 50 degrees and most preferably in a range of about 25 degrees to about 35 degrees. The second seam angle Φ_{S2} is preferably in a range of about 10 degrees to about 50 degrees and most preferably in a range of about 25 degrees to about 35 degrees. The side seam 830 may optionally be configured so that the first and second seam angles Φ_{S1}, Φ_{S2} may be substantially equal.

The garment 800 includes an adjustment arrangement that includes a pair of adjustment tabs 880 that include the respective side seams 830 on either side of the garment and that extend laterally outward from the inner edges 839 of the side seams 830. The adjustment tabs 880 are configured so that they can be grasped and pulled laterally outward and then toward the first main body waist portion 148 (or the second main body waist portion 150) to adjust the fit of the garment 800.

The adjustment tab 880 may be used to separately adjust the fit of the waist encircling portion 806 and each of the leg openings 834. This may be accomplished by drawing the upper portion of the adjustment tab 880 across the side panel 860 and toward the main body 140 in a direction that is roughly perpendicular to the tangent to the curved seam 830 at its intersection with the waist opening edge 831 and drawing a lower portion of the second side panel portion 874 across the side panel 560 and toward the main body 140 in a direction that is roughly perpendicular to the tangent to the curved seam 830 at its intersection with the leg opening edge 833. This approach allows the waist opening 832 to be adjusted without significantly affecting the leg openings 834 and vice versa.

Each adjustment tab 880 may be removably connected to the main body 140 using a fastening arrangement that includes a pair of attachment strips 894. The attachment strips 894 are attached to the adjustment tab 880 so that they extend laterally outward as shown in FIGS. 21 and can be drawn toward the main body 140 as shown in FIG. 22. A gripping member 890 is attached to each attachment strip 894. The gripping member 890 is configured to releasably attach the attachment strip 894 to a receiving portion 192 on the main body 140. The gripping member 890 may comprise a hook surface such as the hook portion of a hook-and-loop fastener, the hook surface being adapted for reversible mating engagement with the receiving portion 192. In another embodiment, the gripping member 890 may comprise a releasable pressure sensitive adhesive for releasably adhering the attachment strip 894 to the receiving portion 192 of the main body 140. A protective release cover may be applied to the adhesive surface to protect the adhesive surface from contamination prior to use. The release cover may be removed just prior to use of the adjustment tabs 880 to adjust the fit of the garment 800.

As previously described, the attachment strips used in conjunction with the present invention may comprise cloth, film, nonwoven material, or any other suitable material. Although the illustrated embodiment includes two attachment strips 894, any number may be used. One or more attachment strips 894 are preferably positioned adjacent the lateral tab edge 886 of the adjustment tab 880. An attachment strip 894 may be positioned adjacent the upper tab edge 882 for securing an adjustment of the waist opening 832. Alternatively or in addition, an attachment strip 894 may be positioned adjacent the lower tab edge 884 for securing an adjustment of one of the leg openings 834.

The attachment strips 894 serve to secure the adjustment tabs 880 in place while maintaining tension on the side seams 830. In an embodiment having two attachment strips 894 extending from the corners of the adjustment tab 880, this may result in a fastened configuration such as that shown in FIG. 22. The fastened configuration of FIG. 22 has the effect of snugging the garment in place against the body of the wearer. It also has the effect of reducing the circumference of the waist opening 832 and associated leg opening 834. It will be understood that the use of the attachment strips 894 eliminates the need for the adjustment tabs 880 to be extendible as far as the main body. This reduces the material required to form the adjustment tabs 880.

It will be understood that other absorbent garment embodiments may be constructed from the unfinished absorbent garment 20, including, for example, embodiments having side seams with a straight portion adjacent the waist opening and an angled portion adjacent the leg opening and embodiments that have a substantially vertical straight portion adjacent the waist opening and a curved portion adjacent the leg opening or vice versa.

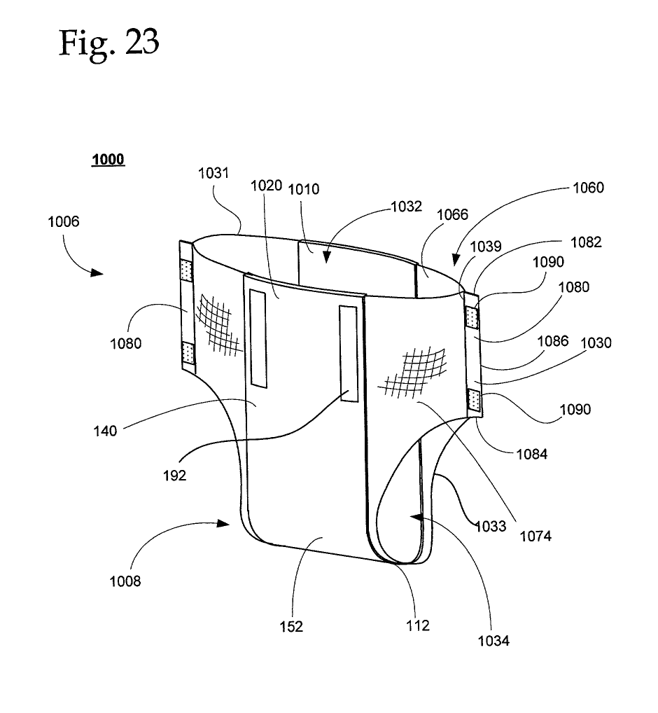

FIGS. 23-25 illustrate an absorbent garment 1000 that may be manufactured from the unfinished garment 10 of FIG. 4. The absorbent garment 1000 has a waist-encircling region 1006 and a crotch region 1008. The waist-encircling region may include a first waist portion 1010, disposable adjacent to the back waist area of a wearer's body, and a second waist portion 1020, disposable adjacent to the front waist area of a wearer's body. The first and second waist portions 1010, 1020 may therefore correspond to the back and front of the wearer's body, respectively. The first and second waist portions 1010, 1020 are joined to one another at side seams 1030 to form the waist encircling portion 1006 and to define a waist opening edge 1031 surrounding a waist opening 1032 and leg opening edges 1033 surrounding two leg openings 1034.

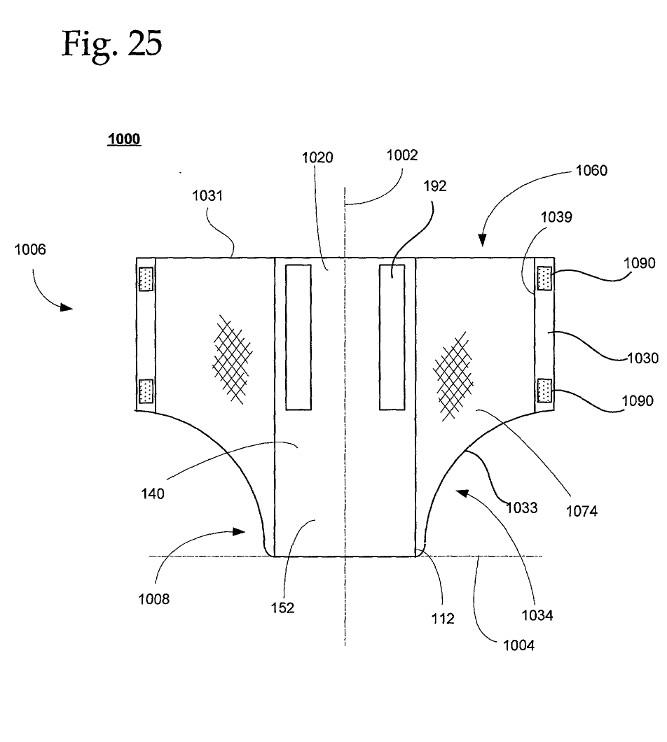

FIGS. 23 and 25 illustrate the garment 1000 as it would appear when initially donned by the wearer. The garment 1000 is shown with and will be described relative to first and second orthogonal axes 1002, 1004. The first axis 1002 may be referred to as the vertical axis 1002 and the second axis 1004 may be referred to as the lateral axis 1004. It will be understood by those having ordinary skill in the art that the vertical axis 1002 corresponds generally to a head-to-toe axis of a standing wearer and that the lateral axis 1004 corresponds generally to a side-to-side axis of a standing wearer.

As shown in FIGS. 23-25, the absorbent garment 1000 includes a main body 140 that is substantially similar to that of the other absorbent garments of the invention. The main body 140 has a central main body portion 152 that connects first and second main body waist portions 148, 150. The first and second main body waist portions 148, 150 are connected on each lateral side of the garment by a side panel 1060. Each side panel 1060 preferably has a first side portion 1066 attached to a lateral edge 168 of the main body 140 adjacent the first main body waist portion 148 and a portion of the central main body portion 152. Each side panel 1060 also preferably has a second side portion 1074 attached to the main body lateral edge 168 adjacent the second main body waist portion 150 and a portion of the central main body portion 152. The first and second side portions 1066, 1074 are joined at a side seam 1030. The first and second side seam portions 1066, 1074 are joined and the side seam 1030 is formed with the first and second side seam portions 1066, 1074 in a fully stretched condition. The side seams 1030 of the garment 1000 are formed in a generally straight line that may be substantially parallel to the vertical axis 1002.

The first and second main body waist portions 148, 150 combine with the side panels 1060 to form the waist opening edges 1031 and the waist opening 1032. The central main body portion 152 combines with the side panels 1060 to form the leg opening edges 133 and the leg openings 1034. The side panels 1060 are preferably formed from an elastic or elasticized material to allow the waist opening 1032 and the leg openings 1034 to expand and contract to provide a secure, leak-free fit.

The garment 1000 includes an adjustment arrangement configured to allow the adjustment of the side panels 1060 to better conform to the body shape of the wearer. The adjustment arrangement preferably includes a pair of adjustment tabs 1080 that include the respective side seams 1030 on either side of the garment and that extend laterally outward from the inner edges 1039 of the side seams 1030. The adjustment tabs 1080 are configured so that they can be grasped and pulled outward and then toward the first main body waist portion 148 (or the second main body waist portion 150) to adjust the fit of the garment 1000. In the illustrated embodiment, the adjustment tabs 1080 are coextensive with the side seams 1030, thus minimizing the amount of material required to form the side panels 1060 and the adjustment tabs 1080. It will be understood, however, that the adjustment tabs 1080 may extend laterally outward beyond the outer edges of the side seams 1030.

The adjustment tabs 1080 are configured so that they may be removably connected to the main body 140 using a fastening arrangement. The fastening arrangement may include one or more gripping members 1090 attached directly to the side seams 1030 as shown in FIGS. 23-25. The gripping members 1090 are configured to releasably attach the adjustment tabs 1080 to a receiving portion 192 on the main body 140. The gripping members 1090 may each comprise a hook surface such as the hook portion of a hook-and-loop fastener, the hook surface being adapted for reversible mating engagement with the receiving portion 192. The receiving portion 192 may include a corresponding loop surface such as the loop portion of a hook-and-loop portion. Alternatively, some or all of the backsheet used to form the main body 140 may be formed using a material that is grippable by the hook surface. In another embodiment, the gripping member 1090 may comprise a releasable pressure sensitive adhesive for releasably adhering the adjustment tab 1080 to the receiving portion 192 of the main body 140. A protective release cover may be applied to the adhesive surface to protect the adhesive surface from contamination prior to use. The release cover may be removed just prior to use of the adjustment tabs 1080 to adjust the fit of the garment 1000.

As shown in FIGS. 23-25, a gripping member 1090 may be positioned adjacent an upper tab edge 1082 for securing an adjustment of the waist opening 1032. Alternatively or in addition, a gripping member 1090 may be positioned adjacent a lower tab edge 1084 for securing an adjustment of one of the leg openings 1034. In an alternative embodiment, a single elongated gripping member may be disposed on the side seam 1030 that extends from the area adjacent the upper tab edge 1082 to the area adjacent the lower tab edge 1084.

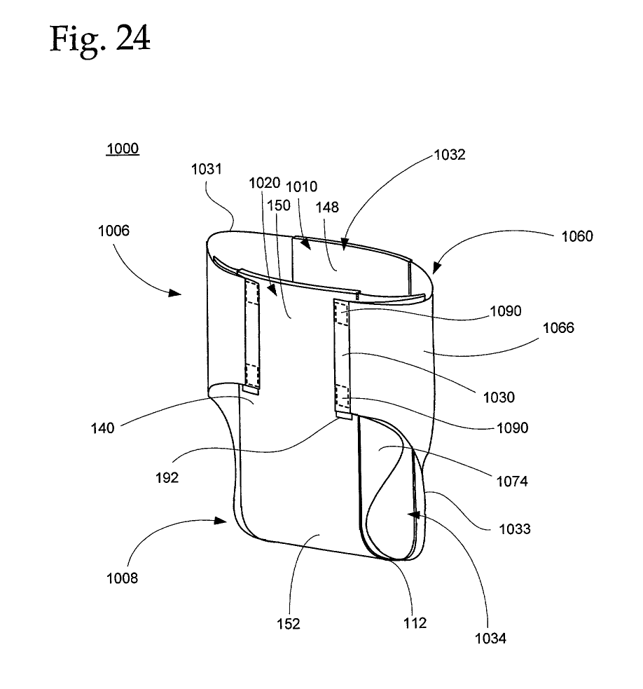

The fastening arrangement serves to secure the adjustment tabs 1080 in place while maintaining tension on the side seams 1030. In an embodiment having two gripping members 1090 at the corners of the adjustment tab 1080, this may result in a fastened configuration such as that shown in FIG. 24. In the fastened configuration of FIG. 24, the first side portion 1066 is placed in tension while the second side portion is folded back in a direction that is roughly perpendicular to the side seam 1030. This has the effect of snugging the garment in place against the body of the wearer. It also has the effect of reducing the circumference of the waist opening 1032 and associated leg opening 1034.

It will be understood by those of ordinary skill in the art that garments having other side seam configurations may have gripping members disposed directly on the side seam. These may include garments having angled straight portions such as those shown in FIGS. 9 and 11 and curved seams such as the those shown in FIG. 15.

The various parts of the absorbent garments of the present invention can be attached to one another or associated with one another to form a structure that preferably maintains its shape during the useful life of the garment. As used herein, the terms “attached,” “joined,” “associated,” and similar terms encompass configurations whereby a first part is directly joined to a second part by affixing the first part directly to the second part, by indirectly joining the first part to the second part through intermediate members, and by fixing the relative positions of various parts by capturing parts between other parts. Those skilled in the art will appreciate that various methods or combinations of methods may be used to securely join the respective parts of the garments to one another.

Due to the wide variety of backing and liner sheet construction and materials currently available, the invention is not intended to be limited to any specific materials or constructions of the components useful in the present invention. The backsheet 144 preferably is made from any suitable pliable liquid-impervious material known in the art. Typical backsheet materials include films of polyethylene, polypropylene, polyester, nylon, and polyvinyl chloride and blends of these materials. For example, the backsheet can be made of a polyethylene film having a thickness in the range of 0.02-0.04 mm. The backsheet 144 may be pigmented with, for example, titanium dioxide, to provide the garment with a pleasing color or to render the backsheet 144 opaque enough that exudates being contained by the garment are not visible from outside the garment. In addition, the backsheet 144 may be formed in such a manner that it is opaque, for example, by using various inert components in the polymeric film and then biaxially stretching the film. Other backsheet materials will be readily apparent to those skilled in the art. The backsheet 144 preferably has sufficient liquid imperviousness to prevent any leakage of fluids. The required level of liquid imperviousness may vary between different locations on the garment.

The backsheet 144 may further comprise separate regions having different properties. In a preferred embodiment, portions of the backsheet 144 are air-permeable to improve the breathability, and therefore comfort, of the garment. The different regions may be formed by making the backsheet 144 a composite or laminate of different sheet materials, chemical treatment, heat treatment, or other processes or methods known in the art. Some regions of the backsheet 144 may be fluid pervious. In certain embodiments of the invention, the backsheet 144 may be fluid impervious in the central main body portion 152, but fluid pervious in portions of the first and second main body waist portions 148, 150.

The moisture-pervious topsheet 142 can be comprised of any suitable relatively liquid-pervious material known in the art that permits passage of liquid there through. Non-woven liner sheet materials are exemplary because such materials readily allow the passage of liquids to the underlying absorbent laminate core 146. Examples of suitable liner sheet materials include non-woven spunbond or carded webs of polypropylene, polyethylene, nylon, polyester and blends of these materials.

The backsheet 144 may be covered with a fibrous, nonwoven fabric such as is disclosed, for example, in U.S. Pat. No. 4,646,362 issued to Heran et al., the disclosure of which is hereby incorporated by reference in its entirety and in a manner consistent with this disclosure. Materials for such a fibrous outer liner include a spun-bonded nonwoven web of synthetic fibers such as polypropylene, polyethylene or polyester fibers; a nonwoven web of cellulosic fibers, textile fibers such as rayon fibers, cotton and the like, or a blend of cellulosic and textile fibers; a spun-bonded nonwoven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. Alternatively, the backsheet 144 may comprise three panels wherein a central poly backsheet panel is positioned closest to absorbent laminate core 146 while outboard non-woven breathable side backsheet panels are attached to the side edges of the central poly backsheet panel. Alternatively, the backsheet 144 may be formed from microporous poly coverstock for added breathability. The image of the invention can suitably be formed on any of these materials, including the fibrous, non-woven outer cover sheet.

The topsheet 142 may be formed of three separate portions or panels. Those skilled in the art will recognize, however, that topsheet 142 need not be made of three separate panels, and that it may be comprised of one unitary item. A first topsheet panel (not shown) may comprise a central topsheet panel formed from preferably a liquid-pervious material that is either hydrophobic or hydrophilic. The central topsheet panel may be made from any number of materials, including synthetic fibers (e.g., polypropylene or polyester fibers), natural fibers (e.g., wood or cellulose), apertured plastic films, reticulated foams and porous foams to name a few. One preferred material for a central topsheet panel or entire topsheet 142 is a cover stock of single ply non-woven material which may be made of carded fibers, either adhesively or thermally bonded, perforated plastic film, spunbonded fibers, or water entangled fibers, which generally weigh from 0.3-0.7 oz./sq. yd. and have appropriate and effective machine direction and cross-machine direction strength suitable for use as a baby diaper cover stock material. The central topsheet panel preferably extends from substantially the second waist portion 120 to the first waist portion 110, or a portion thereof.

A second and third topsheet panel (e.g., outer topsheet panels, not shown), in this alternative embodiment may be positioned laterally outside of the central topsheet panel. The outer topsheet panels preferably are substantially liquid-impervious and hydrophobic, preferably at least in the crotch area. The outer edges of the outer topsheet panels may substantially follow the corresponding outer perimeter of the backsheet 144. The material for the outer topsheet portions or panels preferably is polypropylene and can be woven, non-woven, spunbonded, carded or the like, depending on the application.

At the point of connection between the outer topsheet panels and the central topsheet panel, inner edges of the outer topsheet portions or panels may extend upwardly to form waste containment flaps (not shown). The waste containment flaps preferably are formed of the same material as the outer topsheet portions or panels, as in the embodiment shown. The waste containment flaps may be treated with a suitable surfactant to modify their hydrophobicity/hydrophilicity as desired, and they may be treated with skin wellness ingredients to reduce skin irritation. Alternatively, the waste containment flaps may be formed as separate elements and then attached to the body side liner.

The waste containment flaps preferably include a portion that folds over onto itself to form a small enclosure. At least one, and depending on the size of the enclosure sometimes more than one, elastic member may be secured in the enclosure in a stretched condition. As is known in the art, when the flap elastic attempts to assume the relaxed, unstretched condition, the waste containment flaps rise above the surface of the central topsheet portion or panel.

The topsheet 142 may be made of any suitable relatively liquid-pervious material currently known in the art or later discovered that permits passage of a liquid there through. Examples of suitable topsheet materials include nonwoven spun-bonded or carded webs of polypropylene, polyethylene, nylon, polyester and blends of these materials, perforated, apertured, or reticulated films, and the like. Nonwoven materials are exemplary because such materials readily allow the passage of liquids to the underlying absorbent core 146. The topsheet 142 preferably comprises a single-ply nonwoven material that may be made of carded fibers, either adhesively or thermally bonded, spunbonded fibers, or water entangled fibers, which generally weigh from 0.3-0.7 oz./sq. yd. and have appropriate and effective machine direction (longitudinal) and cross-machine (lateral) direction strength suitable for use as a topsheet material for the given application. The present invention is not intended to be limited to any particular material for the topsheet 142, and other topsheet materials will be readily apparent to those skilled in the art.

The topsheet 142 may further comprise several regions having different properties. In one embodiment of the present invention, the laterally distal portions of the topsheet 142, especially those used to make the outer topsheet panels preferably are substantially fluid impervious and hydrophobic, while the remainder of the topsheet 142 (e.g., central topsheet panel) is hydrophilic and fluid pervious. Different topsheet properties, such as fluid perviousness and hydrophobicity, may be imparted upon the topsheet 142 by treating the topsheet 142 with adhesives, surfactants, or other chemicals, using a composite of different materials, or by other means. The topsheet 142 may also be made from a laminate of overlaid sheets of material. The topsheet 142 also may be treated in specific areas like the crotch region, with skin wellness ingredients such as aloe, vitamin E, and the like.

As noted elsewhere herein, the topsheet 142 and backsheet 144 may be substantially coterminous, or they may have different shapes and sizes. The particular design of the topsheet 142 and backsheet 144 may be dictated by manufacturing considerations, cost considerations, and performance considerations. Preferably, the topsheet 142 is large enough to completely cover the absorbent laminate core 146, and the backsheet 144 is large enough to prevent leakage from the garment. The design of topsheet 142 and backsheet 144 is known in the art, and a skilled artisan will be able to produce an appropriate topsheet 142 and an appropriate backsheet 144 without undue experimentation.

The topsheet 142 and the backsheet 144 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesive, such as Cycloflex as sold by National Starch, a corporation headquartered in Bridgewater, N.J., is used to join the topsheet 142 to the backsheet 144. The particular joining method may be dictated by the types of materials selected for the topsheet 142 and backsheet 144.

As mentioned above, the absorbent garments preferably are provided with containment elastics 154 extending through the central main body portion 152, adjacent the crotch edge. The absorbent garments of the invention also preferably are provided with waist elastic material optionally in the first and second waist portions, 110, 120, respectively, to enable and assist in stretching around the wearer. The waist elastics may be similar structures or different to impart similar or different elastic characteristics to the first and second main body waist portions 148, 150 of the garment. In general, the waist elastics may preferably comprise foam strips positioned at the first and second main body waist portions 148, 150, respectively. Such foam strips preferably are about ½ to about 1½ inches wide and about 3-6 inches long. The foam strips preferably are positioned between the topsheet 142 and the backsheet 144. Alternatively, a plurality of elastic strands may be employed as waist elastics rather than foam strips. The foam strips preferably are comprised of polyurethane, but can be any other suitable material that decreases waist band roll over, reduces leakage over the waist ends of the absorbent garment, and generally improve comfort and fit. The first and optional second waist foam strips preferably are stretched 50-150%, preferably 100% more than their unstretched dimension before being adhesively secured between the backsheet 144 and topsheet 142 or before being secured between two materials, preferably non-woven materials.

The lateral edges of the main body are preferably provided with an adjacent leg elastic containment system including containment elastics 154. In the preferred embodiment, three strands of elastic threads are positioned to extend adjacent to the leg openings 134 between the topsheet 142 and the backsheet 144. Any suitable elastomeric material exhibiting at least an elongation (defined herein as (L_{S}-L_{R})/L_{R} where L_{S} is the stretch length of an elastic element and L_{R} is retracted length, multiplied by 100 to obtain percent elongation) in the range of 5%-350%, preferably in the range of 200%-300%, can be employed for the containment elastics 154. The containment elastics 154 may be attached to the absorbent garment in any of several ways which are known in the art. For example, the containment elastics 154 may be ultrasonically bonded, heat/pressure sealed using a variety of bonding patterns, or glued to the garment. Various commercially available materials can be used for the containment elastics 154, such as natural rubber, butyl rubber or other synthetic rubber, urethane, elastomeric materials such as LYCRA (DuPont), GLOSPAN (Globe) or SYSTEM 7000 (Fulflex).

As stated previously, the invention has been described in connection with a diaper. The invention, however, is not intended to be limited to application only in diapers. Specifically, the fastening arrangements and absorbent cores of the described embodiments may be readily adapted for use in other absorbent garments besides diapers, including, but not limited to, training pants, feminine hygiene products and adult incontinence products. Indeed, given the enhanced thermal transmittance, the absorbent garments of the present invention are particularly suitable for use in adult incontinence products and feminine hygiene products.

The underlying structure beneath the topsheet 142 may include, depending on the diaper construction, various combinations of elements, but in each embodiment, it is contemplated that the absorbent garment will preferably include an absorbent core 146. For example, additional layers may be disposed between the topsheet 142 and absorbent core 146, and/or other additional layers may be disposed between these layers, or between absorbent core 146 and backsheet 144. The additional layer(s) may include a fluid transfer layer, a fluid handling layer, a storage layer, a wicking layer, a fluid distribution layer, and any other layer(s) known to those having ordinary skill in the art.

Although the absorbent core 146 depicted in FIGS. 4 and 8 has a substantially rectangular cross-sectional and plan view shape, other shapes may be used, such as a “T” shape or an hourglass shape. The shape of the absorbent core 146 may be selected to provide the greatest absorbency with a reduced amount of material. The absorbent core may be associated with the topsheet 142, backsheet 144, or any other suitable part of the garment by any method known in the art, in order to fix the absorbent core 146 in place. In addition to the respective layers in the absorbent core 146, as will be described in greater detail hereinafter, the overall absorbent core 146 may be enclosed within a tissue wrapping, as disclosed in U.S. Pat. No. 6,068,620, the disclosure of which is incorporated by reference herein in its entirety. Skilled artisans are capable of designing and wrapping a suitable absorbent core 146 of the invention, using the guidelines provided herein.

Any suitable absorbent material may be used for absorbent core 146. Absorbent cores containing a mixture of fibrous material and super absorbent polymers (SAP) are well known in the art and described, for example, in U.S. Pat. No. 5,281,207, to Chmielewski, and U.S. Pat. No. 5,863,288, to Baker, the disclosures of each of which are herein incorporated by reference in their entirety and in a manner consistent with this disclosure. The fibrous material can be any fibrous material, preferably one that is capable of absorbing fluids and capable of retaining SAP particles within its matrix. Preferred fibrous materials may be selected from cellulose acetate fibers, rayon fibers, Courtauld's LYOCELL fibers, polyacrylonitrile fibers, surface-modified (hydrophilic) polyester fibers, surface-modified polyolefin/polyester bicomponent fibers, surface-modified polyester/polyester bicomponent fibers, cotton fibers, or blends thereof. In addition, rayon, Courtauld's LYOCELL, polyacrylonitrile, cotton fibers and cotton linters are alternatively preferred. The remaining fibers, surface-modified polyolefin/polyester bicomponent fibers, and surface-modified polyester/polyester bicomponent fibers are also believed to be effective fibrous materials for use in the invention.

Any SAP now known or later discovered may be used in absorbent core 146, so long as it is capable of absorbing liquids. Useful SAP materials are those that generally are water-insoluble but water-swellable polymeric substance capable of absorbing water in an amount that is at least ten times the weight of the substance in its dry form. In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims and equivalents thereof.

What is claimed is:

1. An absorbent article comprising:
a main body having a first main body waist portion, a second main body waist portion and a central main body portion disposed between the first and second main body waist portions, the main body having orthogonal vertical and lateral axes;
a pair of side panels, each side panel connecting the first main body waist portion to the second main body waist portion, the first main body waist portion, the second main body waist portion and the side panels collectively defining a waist opening edge and two leg opening edges, each side panel being formed from a first side portion extending laterally outward from the first main body waist portion and a second side portion extending laterally outward from the second main body waist portion, the first side portion being attached to the second side portion by a side seam intersecting the waist opening edge at a first seam angle relative to the vertical axis and intersecting one of the leg openings at a second seam angle relative to the vertical axis when the first and second side portions are in a fully stretched condition; and
an adjustment arrangement including at least one adjustment tab associated with one of the side panels, each of the at least one adjustment tab including the side seam of the associated side panel and extending outward therefrom and having a lateral tab edge, the adjustment arrangement also including at least one adjustment tab fastener adapted for removably attaching the at least one adjustment tab to a receiving portion of the main body.

2. An absorbent article according to claim 1 wherein each of the at least one adjustment tab fastener includes a gripping member.

3. An absorbent article according to claim 2 wherein the receiving portion includes at least one fastener attachment member, the fastener attachment member being adapted for reversible mating engagement with the gripping member.

4. An absorbent article according to claim 2 wherein the gripping member is disposed on the adjustment tab, the gripping member being positioned so that the gripping member can engage the receiving portion of the main body when the garment is arranged in an adjusted configuration on a wearer.

5. An absorbent article according to claim 4 wherein the gripping member is disposed on the side seam of the associated side panel.

6. An absorbent article according to claim 2 wherein each of the at least one adjustment tab fastener includes a fastening strip extending laterally outward from the lateral tab edge, the gripping member being disposed on the fastening strip, the fastening strip and the gripping member being adapted so that the gripping member can engage the receiving portion of the main body when the garment is arranged in an adjusted configuration on a wearer.

7. An absorbent article according to claim 1 wherein the fastening arrangement includes a first adjustment tab fastener including a first gripping member disposed on the adjustment tab, the first gripping member being positioned so that the first gripping member can engage the receiving portion of the main body when the garment is arranged in a first adjusted configuration on a wearer, and a second adjustment tab fastener including a second gripping member disposed on the adjustment tab, the second gripping member being positioned so that the second gripping member can engage the receiving portion of the main body when the garment is arranged in a second adjusted configuration on a wearer.

8. An absorbent article according to claim 1 wherein the fastening arrangement includes a first adjustment tab fastener including a first fastening strip extending laterally outward from the lateral tab edge and a first gripping member disposed on the first fastening strip, the first fastening strip and the first gripping member being adapted so that the first gripping member can engage the receiving portion of the main body when the garment is arranged in a first adjusted configuration on a wearer, and a second adjustment tab fastener including a second fastening strip extending laterally outward from the lateral tab edge and a second gripping member disposed on the second fastening strip, the second fastening strip and the second gripping member being adapted so that the second gripping member can engage the receiving portion of the main body when the garment is arranged in a second adjusted configuration on a wearer.

9. An absorbent article according to claim 1 wherein each of the at least one adjustment tab is formed at least in part from the first and second side portions of the associated side panel.

10. An absorbent article according to claim 1 wherein at least one of the first and second side seam angles is in a range from about 10 degrees to about 80 degrees.

11. An absorbent article according to claim 1 wherein the first side seam angle is in a range from about 10 degrees to about 80 degrees.

12. An absorbent article according to claim 1 wherein the first side seam angle is in a range from about 10 degrees to about 50 degrees.

13. An absorbent article according to claim 1 wherein the first side seam angle is in a range from about 25 degrees to about 35 degrees.

14. An absorbent article according to claim 1 wherein the second side seam angle is in a range from about 10 degrees to about 80 degrees.

15. An absorbent article according to claim 1 wherein the second side seam angle is in a range from about 25 degrees to about 35 degrees.

16. An absorbent article according to claim 1 wherein the second side seam angle is in a range from about 25 degrees to about 35 degrees.

17. An absorbent article according to claim 1 wherein at least a portion of the side seam is curved when the first and second side portions are in a fully stretched condition.

18. An absorbent article according to claim 1 wherein at least a portion of the side seam adjacent the waist opening edge is curved when the first and second side portions are in a fully stretched condition.

19. An absorbent article according to claim 1 wherein at least a portion of the side seam adjacent the leg opening edge is curved when the first and second side portions are in a fully stretched condition.

20. An absorbent article comprising:
a main body having a first main body waist portion, a second main body waist portion and a central main body portion disposed between the first and second main body waist portions, the main body having orthogonal vertical and lateral axes;
a pair of side panels, each side panel connecting the first main body waist portion to the second main body waist portion, the first main body waist portion, the second main body waist portion and the side panels collectively defining a waist opening edge and two leg opening edges, each side panel being formed from a first side portion extending laterally outward from the first main body waist portion and a second side portion extending laterally outward from the second main body waist portion, the first side portion being attached to the second side portion by a side seam intersecting the waist opening edge at a first seam angle relative to the vertical axis and intersecting one of the leg openings at a second seam angle relative to the vertical axis when the first and second side portions are in a fully stretched condition, at least a portion of the side seam being curved when the first and second side portions are in a fully stretched condition; and
an adjustment arrangement including at least one adjustment tab associated with one of the side panels, each of the at least one adjustment tab including the side seam of the associated side panel and extending outward therefrom and having a lateral tab edge, the adjustment arrangement also including at least one adjustment tab fastener adapted for removably attaching the at least one adjustment tab to a receiving portion of the main body.

21. An absorbent article according to claim 20 wherein each of the at least one adjustment tab fastener includes a gripping member and wherein the receiving portion includes at least one fastener attachment member, the fastener attachment member being adapted for reversible mating engagement with the gripping member.

22. An absorbent article according to claim 20 wherein each of the at least one adjustment tab fastener includes a fastening strip extending laterally outward from the lateral tab edge, a gripping member being disposed on the fastening strip, the fastening strip and the gripping member being adapted so that the gripping member can engage the receiving portion of the main body when the garment is arranged in an adjusted configuration on a wearer.

23. An absorbent article according to claim 20 wherein each of the at least one adjustment tab is formed at least in part from the first and second side portions of the associated side panel.

24. An absorbent article according to claim 20 wherein at least one of the first and second side seam angles is in a range from about 10 degrees to about 80 degrees.

25. An absorbent article according to claim 20 wherein at least one of the first and second side seam angles is in a range from about 10 degrees to about 50 degrees.

26. An absorbent article according to claim 20 wherein at least one of the first and second side seam angles is in a range from about 25 degrees to about 35 degrees.

27. An absorbent article according to claim 20 wherein at least a portion of the side seam adjacent the waist opening edge is curved.

28. An absorbent article according to claim 20 wherein at least a portion of the side seam adjacent the leg opening edge is curved.
